SEQUENCE LISTING (14 sequences)

SEQ ID NO: 1 - RNA, 24 nt, E. coli
auguggauuggcgauaaaaaacaa

SEQ ID NO: 2 - DNA, 35 bp, Artificial Sequence
  LacZ Probe
gttgttttttaucgccaauccacauctgtgaaaga

SEQ ID NO: 3 - RNA, 35 nt, Bacillus anthracis
aucuuuagaagcauuaucugaagauaagaaaaaaa

SEQ ID NO: 4 - DNA, 37 bp, Artificial Sequence
  BA probe
gatttttttcuuaucuucagauaatgcttctaaagat

SEQ ID NO: 5 - RNA, 36 nt, Avian influenza virus
aaucuucuugaaaauuugcagaccuaccaaaaacga

SEQ ID NO: 6 - DNA, 37 bp, Artificial Sequence
  BF Probe
tcgtttttgguaggucugcaaaauuuucaagaagatt

SEQ ID NO: 7 - RNA, 36 nt, Avian influenza virus
cauuuaucgucgccuuuaaauacgguuugaaaagag

SEQ ID NO: 8 - DNA, 35 bp, Artificial Sequence
  AF Probe
ctcttttcaaaccguauuuaaggcgacgataaatg

SEQ ID NO: 9 - DNA, 35 bp, Artificial Sequence
  Biotin-labeled LacZ DNA
gttgttttttatcgccaatccacatctgtgaaaga

SEQ ID NO: 10 - RNA, 21 nt, Dengue virus
ggaagcuguacgcauggggua

SEQ ID NO: 11 - DNA, 21 bp, Artificial Sequence
  Match (M) probe
taccccatgcguacagcuucc

SEQ ID NO: 12 - DNA, 21 bp, Artificial Sequence
  Mismatch-1 (Mis-1) probe
tacgccatgcguacagcuucc

SEQ ID NO: 13 - DNA, 21 bp, Artificial Sequence
  Mismatch-2 (Mis-2) probe
tacaccgtgcguacagcuucc

SEQ ID NO: 14 - DNA, 21 bp, Artificial Sequence
  Mismatch-3 (Mis-2) probe
tatgccacgcguacagcuucc

(19) United States
(12) Patent Application Publication
(10) Pub. No.: US 2016/0068894 A1
(43) Pub. Date: Mar. 10, 2016

(54) RNA Microchip Detection Using Nanoparticle-Assisted Signal Amplification

(71) Applicant: GEORGIA STATE UNIVERSITY RESEARCH FOUNDATION, INC., Atlanta, GA (US)

(72) Inventor: Zhen Huang, Marietta, GA (US)

(21) Appl. No.: 14/782,346

(22) PCT Filed: Apr. 4, 2014
(86) PCT No.: PCT/US14/33062

§ 371 Date: Oct. 5, 2015

Related U.S. Application Data
(60) Provisional application No. 61/808,447, filed on Apr. 4, 2013.

(51) Int. Cl. C12Q 1/68

(52) U.S. Cl. CPC C12Q 1/6837

(57) ABSTRACT

Disclosed are methods and materials for detecting RNA in a sample. In some forms, the method involves (a) bringing into contact the sample and a probe array, (b) bringing into contact the probe array and a ribonuclease specific for RNA/DNA hybrids (such as RNase H), (c) bringing into contact the probe array, labeled nucleotides, and a nucleic acid polymerase capable of extending a RNA strand using a DNA template and capable of incorporating the labeled nucleotides in the extension from the RNA strand (such as Klenow fragment DNA polymerase), and (d) detecting the labeled nucleotides in the extended nucleic acid strand. The probe array comprises one or more chimeric probes. The chimeric probes comprise a DNA region and a RNA region, where the DNA region and the RNA region are contiguous and where the DNA region is 5′ of the RNA region. The chimeric probe can also include a second DNA region. The second DNA region can also be contiguous with the RNA region and can be 3′ of the RNA region.

### CROSS—REFERENCE TO RELATED APPLICATIONS

This application claims benefit of U.S. Provisional Application No. 61/808,447, filed Apr. 4, 2013. Application No. 61/808,447, filed Apr. 4, 2013, is hereby incorporated herein by reference in its entirety.

### REFERENCE TO SEQUENCE LISTING

The Sequence Listing submitted Apr. 4, 2014 as a text file named “GSURF_{—}2013_{—}17_PCT_Sequence_Listing.txt,” created on Apr. 4, 2014, and having a size of 4,889 bytes is hereby incorporated by reference pursuant to 37 C.F.R. §1.52(e)(5).

### FIELD OF THE INVENTION

The disclosed invention is generally in the field of nucleic acid detection and analysis and specifically in the area of RNA detection and analysis.

### BACKGROUND OF THE INVENTION

Natural pandemics (for example, West Nile virus (WNV) and flu pandemics) (Pripuzova et al., PLoS One, 7, e43246 (2012); Wheeler et al., Am J Trop Med Hyg, 87, 559 (2012); Brault et al., J Med Entomol, 49, 939 (2012); Kesavaraju et al., Am J Trop Med Hyg, 87, 359 (2012)) and food poisoning outbreaks (such as Escherichia coli O157:H7) are deadly threats to human health and lives. Rapid and accurate detection of these human pathogens at the point of care is essential for proper medical treatment and saving lives. Viral and bacterial pathogen detection for infectious disease rapid diagnosis is a major healthcare challenge. For example, H1N1 influenza, WNV, bacillus anthracis and pathogenic E. coli need to be rapidly detected in order to minimize the scope of the outbreak and the deadly impact of the outbreak. Culture-based methods are time-consuming and need normally 2-3 days before obtaining conclusions (March and Ratnam, J Clin Microbiol, 23, 869 (1986)). Antigen-antibody-based immunological assay (i.e., ELISA) cannot effectively differentiate the subtle differences between different strains, though it is rapid and robust. On the other hand, the nucleic acid-based detection methods, such as real-time polymerase chain reaction (PCR) and cDNA arrays (Bustin, J Mol Endocrinol, 25, 169 (2000); Call, Crit Rev Microbiol, 31, 91 (2005); Vora et al., Molecular and cellular probes, 22, 294 (2008)), can detect a single nucleotide difference and offer high detection specificity. However, they don't detect RNAs directly, and reverse transcription (RT) and multiple steps are required. PCR amplification may cause false positive results due to the lack of strict contamination control. Many other strategies, such as nucleic acid sequence-based amplification (Zhao et al., J Clin Microbiol, 47, 2067 (2009)), mass spectroscopy (Li et al., Anal Chem, 82, 3399 (2010)), and rolling circle amplification (Murakami et al., Nucleic Acids Res, 40, e22 (2012)), have been developed for DNA detection.

Furthermore, current methods for direct RNA detection, such as Northern blot, RNase protection assay, microRNA profiling, and direct RNA sequencing (Nelson et al., Nat Methods, 1, 155 (2004); Sandelin et al., Nat Rev Genet, 8, 424 (2007); Ozsolak et al., Nature, 461, 814 (2009)), are labor-intensive, time-consuming, costly and/or instrument-intensive. These approaches are not well-suited for rapid and accurate RNA detection, especially for point-of-care diagnosis and field applications. Recently, the multiplex RNA biosensor based on surface plasmon resonance (SPR) (Fang et al., J Am Chem Soc, 128, 14044 (2006); Lee et al., Langmuir, 22, 5241 (2006)) and the microRNA microarray based on locked nucleic acid (LNA) and Au-nanoparticle conjugates have been demonstrated (Castoldi et al., RNA, 12, 913 (2006)). However, the RNA sample preparation and convenient detection are still complicated and time-consuming, which are major challenges in direct RNA detection for the point-of-care pathogen and disease diagnosis.

It is an object of the present invention to provide methods and compositions for rapid and sensitive detection of RNA without the need for nucleic acid amplification.

It is another object of the present invention to provide methods and compositions for easy detection of RNA without the need for equipment that is expensive and/or complex to operate.

It is another object of the present invention to provide methods and compositions for detection of pathogens.

### BRIEF SUMMARY OF THE INVENTION

Disclosed are methods and materials for detecting RNA in a sample. In some forms, the method involves (a) bringing into contact the sample and a probe array, (b) bringing into contact the probe array and a ribonuclease specific for RNA/DNA hybrids (such as RNase H), (c) bringing into contact the probe array, labeled nucleotides, and a nucleic acid polymerase capable of extending a RNA strand using a DNA template and capable of incorporating the labeled nucleotides in the extension from the RNA strand (such as Klenow fragment DNA polymerase), and (d) detecting the labeled nucleotides in the extended nucleic acid strand. The probe array comprises one or more chimeric probes. The chimeric probes comprise a DNA region and a RNA region, where the DNA region and the RNA region are contiguous and where the DNA region is 5′ of the RNA region. The chimeric probe can also include a second DNA region. The second DNA region can also be contiguous with the RNA region and can be 3′ of the RNA region. If the sample contains an RNA molecule complementary to a nucleotide sequence of at least one of the chimeric probes, the RNA molecule will hybridize with the complementary chimeric probe. In some forms, bringing into contact the sample and a probe array, bringing into contact the probe array and the ribonuclease, and bringing into contact the probe array, labeled nucleotides, and the nucleic acid polymerase (steps (a), (b), and (c)) are carried out simultaneously.

In the method, RNA molecules in the sample can hybridize to chimeric probes having complementary sequence. The portion of an RNA molecule hybridized to the DNA region of the chimeric probe is degraded by the ribonuclease specific for RNA/DNA hybrids. The hybridized RNA molecule can then extended to form an extended nucleic acid strand, where the DNA region of the chimeric probe serves as the template. For labeling, at least one labeled nucleotide is incorporated into the extended nucleic acid strand. The labeled nucleotides comprise a first label. Because of the sequence relationship between the chimeric probe and the RNA molecule that hybridizes to the chimeric probe, detection of the labeled nucleotides in the extended nucleic acid strand indicates the presence of the RNA molecule in the sample.

In some forms of the method, labeling and detection can be enhanced by labeling the first label. This can be accomplished, for example, by bringing into contact the probe array and a label conjugate, where the label conjugate comprises a specific binding molecule and a second label, and where the specific binding molecule binds to the first label. The labeled nucleotides in the extended nucleic acid strand are then detected by detecting the second label. An example of a useful combination of first label and label conjugate are biotin as the first label and a streptavidin-conjugated gold nanoparticle as the label conjugate. In this example, the streptavidin is the specific binding molecule that binds to the first label (biotin) and gold nanoparticle is the second label.

By using another conjugate that can aggregate on or at the site of the second label, the amount of second label at the site is increased, increasing the sensitivity of the detection and making detection easier. This can be accomplished, for example, by bringing into contact the probe array and a detection conjugate, where detection conjugate comprises an aggregator, and where the aggregator mediates aggregation of detection conjugates on the label conjugate. An example of a useful combination of second label and detection conjugate are gold nanoparticle as the second label and a silver nanoparticle as the detection conjugate. In this example, the silver nanoparticle is the aggregator. The silver nanoparticle reacts with the gold nanoparticle to accumulate silver nanoparticles at the site of the gold nanoparticles. The accumulation of the reacted silver nanoparticles can be sufficient to detect with the naked eye.

Rapid and accurate detection of pathogens, such as RNA viruses at the point of care, will allow proper patient treatment and save lives. The disclosed RNA microchip can directly detect RNA without reverse transcription and PCR amplification. The disclosed RNA microchip can use nanoparticle-assisted signal amplification. The disclosed RNA microchip technology is simple and accurate and can differentiate single-nucleotide difference. In some forms, RNA can be sensitively detected within 1 hour, and the signal can be detected by naked eye. The visual readout format and simplicity make the disclosed RNA microchip technology well suited for the rapid and accurate detection of pathogens in clinics and in the field with minimum resources.

The disclosed technology can also be used to detect and analyze any RNA or combination of RNA from single or multiple sources. For example, the technology can be used to detect RNA expression patterns in cells and samples. As another example, the probe array can include one or more of the chimeric probes that are complementary to a nucleotide sequence characteristic of, for example, a virus, a bacteria, or a microbe. Detection of the characteristic nucleotide sequence indicates the presence of the corresponding virus, bacteria, or microbe.

The disclosed methods can be aided by using solid state substrates to which the chimeric probes are immobilized as the probe array. The location of particular chimeric probes allows identification of the RNA molecule detected since the sequence of the chimeric probe at a location where label is detected is known. By including multiple different chimeric probes on a probe array, multiple different RNA molecules can be detected. Related RNA molecules can be detected collectively by grouping different chimeric probes specific to the different RNA molecules at eh same location on the probe array. This can simplify detection of targets that, for example, have variable sequences.

In some forms, the chimeric probes can be stabilized. Stabilized nucleic acids, such as the chimeric probes, can, for example, use modified nucleotides that make the nucleic acid resistant to degradation. Examples of useful modified nucleotides include 2′-O-methyl nucleotides. In some forms, the chimeric probes can also include a 3′-linking group to allow, facilitate, or mediate immobilization of the chimeric probes. For example, the 3′-linking group can be an amino group.

Also disclosed are probe arrays. Probe arrays for use in the disclose methods can comprise one or more of the disclosed chimeric probes. Also disclosed are kits. Kits for use in the disclosed methods can comprise a probe array, labeled nucleotides and a label conjugate, all as disclosed herein. The kits can also and/or alternatively include a ribonuclease specific for RNA/DNA hybrids (such as RNase H), a nucleic acid polymerase capable of extending a RNA strand using a DNA template and capable of incorporating the labeled nucleotides in the extension from the RNA strand (such as Klenow fragment DNA polymerase), or both.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows examples of the disclosed RNA microchip and RNA visual detection using nanoparticle-assisted signal amplification. Shown is a flowchart of RNA rapid and direct detection. RNA probe hybridizes with the target RNA, followed by RNase H digestion and Klenow extension and incorporation of biotin-labeled dNTPs. Streptavidin-conjugated Au-NPs bind to biotins and then convert the biotin labels to the Au-NP labels. Au-NPs catalyze Ag-NP formation and aggregation for visual detection with naked eye (or aided with magnifying glass).

FIG. 2A shows RNA direct detection with single-nucleotide discrimination on the microchip. In order, from top to bottom, the sequences are SEQ ID NO:10, SEQ ID NO:11, SEQ ID NO:12, SEQ ID NO:13, SEQ ID NO:14, nucleotides 1-13 of SEQ ID NO:10, SEQ ID NO:11, SEQ ID NO:15, and SEQ ID NO:11. The size of the signal spot is approximately 75 micron, which is visible by naked eye (or aided with magnify glass). All signal spots are in the similar size on this and other RNA microchips.

FIGS. 2B, 2C, 2D, 2E, and 2F show RNA microchip and RNA visual detection. (B) RNA detection in the absence of biotinated dNTPs (negative control). (C) The absence of RNAs (negative control). (D) Using native dNTPs (negative control). (E) RNA detection in the presence of RNAs and biotinated dNTPs. (F) RNA detection with single-nucleotide discrimination. The signal spots (approximately 75 micron) are visible by naked eye. All signal spots are in the similar size on this and other RNA microchips.

FIGS. 3A, 3B, 3C, 3D, 3E, and 3F show specificity and individual mRNA detection with an example of the disclosed RNA microchip. RNA microchips were immobilized with the RNA probes. BF, AF, LacZ and BA probes detect bird flu (BF) RNA, Avian flu (AF) RNA, LacZ RNA, and Bacillus anthracis (BA) RNA, respectively. The target RNAs were detected by specifically incorporating biotin-labeled dNTPs into the target RNAs. (A) The sample contains BA RNA. (B) The sample contains LacZ and BA RNAs. (C) The sample contains BF, AF and LacZ RNAs. (D) The sample contains all the RNAs. (E) Detection of an individual RNA (LacZ mRNA) in total RNA. Chip I: water (no RNA; negative control); Chip II: the total RNA (containing LacZ) isolated from the IPTG-induced E. coli; Chip III: the total RNA (containing no LacZ) isolated from the glucose-suppressed E. coli. (F) Detection of an individual RNA (LacZ mRNA) via the simple RNA sample preparation by NaOH direct treatment of E. coli. Chip I: water (no RNA; negative control); Chip II and III: the NaOH-treated IPTG-induced and glucose-suppressed E. coli, respectively.

FIG. 4 shows activation of the glass microchip surface as an example of probe immobilization. Surface chemistry enables immobilization of amine modified chimeric probes.

FIGS. 5A, 5B, 5C, 5D, and 5E show sensitive and rapid RNA detection with an example of the disclosed RNA microchip. (A) Sensitive detection. Chip 1-5 contain 0, 5, 15, and 50 fmole of LacZ RNA, respectively. (B) RNA detection via consolidation of the RNase H and Klenow steps. (C) RNA detection via consolidation of the hybridization, RNase H and Klenow steps (15-min incubation for the combined step). (D) Rapid RNA detection (in 45 min, including RNA sample preparation). Biotined-oligo-35.2 (positive control); BA probe (negative control) doesn't detect E. coli RNAs. (E) Detection of E. coli RNA (LacZ RNA) after the IPTG-induced cells were treated with 70% ethanol for 0, 30, 45 and 60 min. Glucose-suppressed E. coli cells (Glu) were used as the negative control.

### DETAILED DESCRIPTION OF THE INVENTION

The disclosed method and compositions may be understood more readily by reference to the following detailed description of particular embodiments and the Example included therein and to the Figures and their previous and following description.

Microarray technology presents a powerful tool for the analysis of specific mRNAs of cell populations. The utility of DNA microarrays is limited by its indirect analysis of RNA, which increases the processing time, as well as multiple biases and artifacts. Direct detection of RNAs is still a challenge, since RNAs are easily degraded and labeling a specific RNA in an RNA mixture is difficult. Disclosed are direct, rapid, specific, easy to use, cost effective and sensitive microchips for, for example, pathogen and viral RNA detection. By use of nucleases, polymerases and nanomaterials, specific RNA can be detected without reverse transcription. In multi-target forms of the method, detection method is specific and can be accomplished in less than an hour. The disclosed technology can be used to detect and analyze specific mRNAs in a wide array of fields, including, for example, infectious diseases diagnosis, food safety, gene expression profiling, and cancer detection.

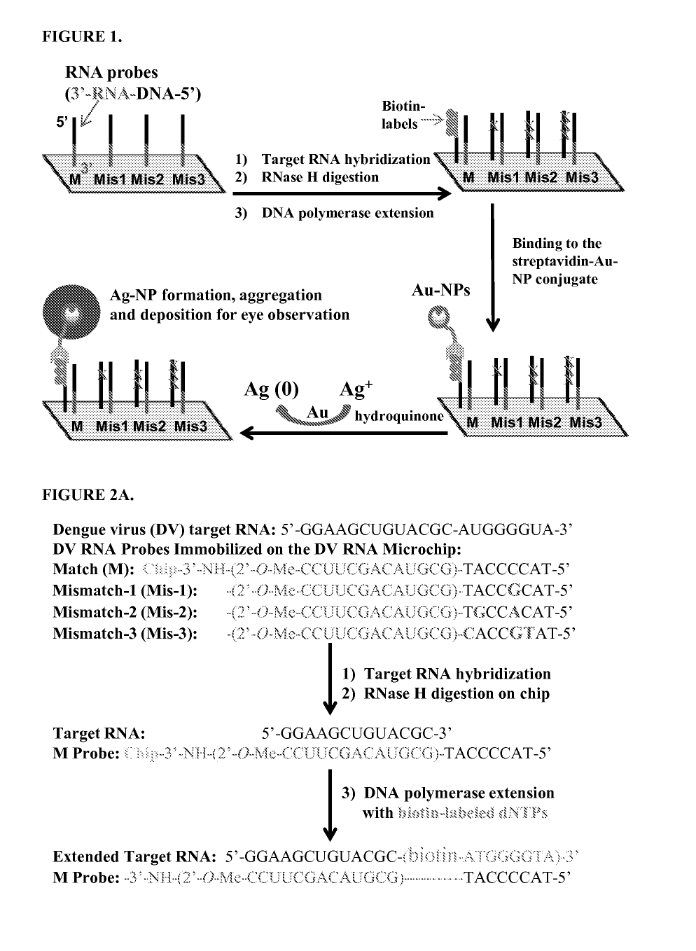

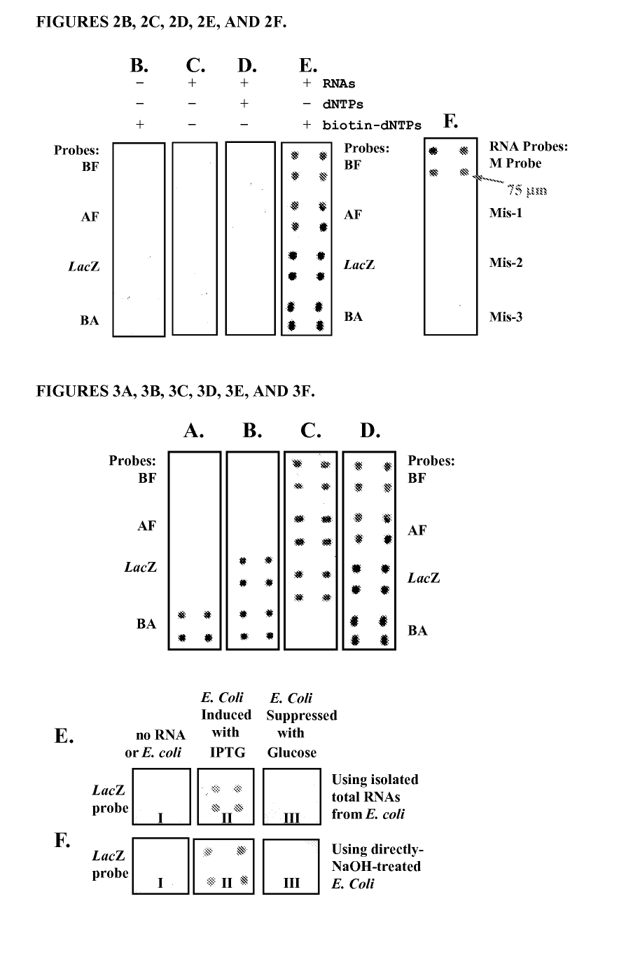

In some forms, the disclosed RNA detection system is simple: after target RNA hybridization to RNA microchip immobilized with RNA probes (Spencer et al., ChemBioChem, 11, 1378 (2010)), the bound RNAs are digested with RNase H and extended with biotin-labeled dNTPs by Klenow DNA polymerase. Using the conjugate of streptavidin and gold nanoparticle (Au-NP), the biotin labels incorporated to the target RNAs are then converted to Au-NP labels. The Au-NP can catalyze the formation of silver nanoparticles (Ag-NP) via the silver staining (Taton et al., Science, 289, 1757 (2000); Cao et al., Biosens Bioelectron, 22, 393 (2006): Qi et al., Anal Bioanal Chem, 398, 2745 (2010); Tang et al., Diagn Microbiol Infect Dis, 65, 372 (2009); Zhou et al., J. Am Chem Soc, 132, 6932 (2010)), allowing the signal amplification up to 100 times. The formed Ag-NPs (FIGS. 1 and 2) aggregate and deposit as dark spots on the RNA probe sites. Finally, an image of an array of these dark spots will form for direct visual observation. Moreover, we found that this RNA microchip can differentiate single nucleotide difference and detect RNA at low fmole level. The RNA detection system is based on our RNA 3′-labeling approach, where a DNA polymerase directly incorporates labeled dNTPs into RNA on a DNA template (Huang and Alsaidi, Analytical Biochemistry, 322, 269 (2003); Alsaidi et al., ChemBioChem, 5, 1136 (2004)). In order to directly detect RNA, we design the functionalized RNAs as the RNA probes. A RNA probe consists of the RNA 3′-region and the DNA 5′-region (FIGS. 1 and 2).

Disclosed are methods and materials for detecting RNA in a sample. In some forms, the method involves (a) bringing into contact the sample and a probe array, (b) bringing into contact the probe array and a ribonuclease specific for RNA/DNA hybrids (such as RNase H), (c) bringing into contact the probe array, labeled nucleotides, and a nucleic acid polymerase capable of extending a RNA strand using a DNA template and capable of incorporating the labeled nucleotides in the extension from the RNA strand (such as Klenow fragment DNA polymerase), and (d) detecting the labeled nucleotides in the extended nucleic acid strand. The probe array comprises one or more chimeric probes. The chimeric probes comprise a DNA region and a RNA region, where the DNA region and the RNA region are contiguous and where the DNA region is 5′ of the RNA region. If the sample contains an RNA molecule complementary to a nucleotide sequence of at least one of the chimeric probes, the RNA molecule will hybridize with the complementary chimeric probe. In some forms, the chimeric probes can also include a 3′-linking group to allow, facilitate, or mediate immobilization of the chimeric probes. For example, the 3′-linking group can be an amino group.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a chimeric probe is disclosed and discussed and a number of modifications that can be made to a number of molecules including the chimeric probe are discussed, each and every combination and permutation of chimeric probe and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Chimeric Probes

Chimeric probes are oligonucleotides that can hybridize to RNA molecules in a sequence-specific manner. Chimeric probes comprise a DNA region and a RNA region, where the DNA region and the RNA region are contiguous and where the DNA region is 5′ of the RNA region. In some forms, the chimeric probe can also include a second DNA region. In some forms, the second DNA region can also be contiguous with the RNA region and can be 3′ of the RNA region. In the disclosed method, chimeric probes are used to capture RNA molecules based on complementary sequences present in the RNA molecules. The DNA region(s) of the chimeric probe allows formation of a RNA/DNA hybrid between the chimeric probe and the RNA molecule of interest. This allows the RNA strand in the DNA/RNA hybrid to be selectively degraded by a DNA/RNA hybrid-specific ribonuclease such as RNase H.

To facilitate the function of the chimeric probe in the disclosed methods, all or a portion of the DNA region(s) contiguous to the RNA region of a chimeric probe and all or a portion of the RNA region contiguous to the DNA region(s) of the chimeric probe are complementary to a contiguous sequence in an RNA molecule of interest. In other words, a nucleotide sequence of the chimeric probe spanning the junction of the RNA region and the DNA region(s) of the chimeric probe is complementary to a sequence in an RNA molecule of interest. This allows a portion of the RNA molecule to hybridize to the RNA region of the chimeric probe and another, contiguous portion of the RNA molecule to hybridize to the DNA region(s) of the chimeric probe. The hybridized portion of the RNA molecule will span the junction of the RNA region and DNA region(s) of the chimeric probe.

Chimeric probes can also include spacers, linkers, linkage groups to attach the chimeric probe to a substrate, and additional nucleic acid regions. These additional components can be used for suitable purposes, such as to aid production or handling of chimeric probes and to aid in immobilizing the chimeric probes on a substrate to form a probe array. Some additional components can be byproducts of the method by which the chimeric probes are made. The nature of these additional components of chimeric probes generally is not critical to function of the chimeric probes in the disclosed methods. All that is required is that the chimeric probe can hybridize to the RNA molecule of interest and that the enzymes of the method can act on the hybrids.

Chimeric probes can be, and preferably are, immobilized on a substrate. Chimeric probes need not be composed of naturally occurring nucleotides. Modified nucleotides, unnatural bases and nucleotide and oligonucleotide analogs can be used. All that is required is that the probe has the general structure described herein and be capable of the interactions and reactions required in the disclosed method. In particular, chimeric probes can be stabilized nucleic acids. As used herein, stabilized nucleic acids are nucleic acids that include one or more modifications relative to natural nucleic acids that render the nucleic acid less prone to degradation or cleavage.

To facilitate or mediate immobilization of the chimeric probes, the chimeric probes can include a 3′-linking group. As used herein, a 3′-linking group is a group or moiety that facilitates or mediates attachment to a substrate. For example, the 3′-linking group can be an amino group.

Chimeric probes preferably include a complementary portion (also referred to as a probe portion (for hybridization to sample fragments) and a linker portion through which the probe portion is coupled to a substrate. These linker portions can have any suitable structure and will generally be chosen based on the method of immobilization or synthesis of the chimeric probes. The linker portion can be made up of or include nucleotides. The linker portions can have any suitable length and preferably are of sufficient length to allow the probe portion to hybridize effectively. For convenience and unless otherwise indicated, reference to the length of chimeric probes refers to the length of the probe portion of the probes. Immobilized chimeric probes are chimeric probes immobilized on a support.

Chimeric probes can be used in sets having a variety of probe sequences, such as a set of probes corresponding to a variety of target RNA molecules. For example, a set of chimeric probes collectively can be complementary to RNA sequences specific for a set of pathogens. Chimeric probes can be used in sets where each probe has the same length. Preferred lengths for the complementary portion of chimeric probes are 15, 16, 17, 18, 19, 20, 21, 22, 23, 24, 25, 26, 27, 28, 29, and 30 nucleotides. The complementary portion of the RNA region of chimeric probes preferably is sufficient to form a stable hybrid with the target RNA molecule when sequence of the RNA molecule in the DNA/RNA hybrid is degraded. Preferred lengths for the complementary portion of the RNA region of chimeric probes are 5, 6, 7, 8, 9, 10, 11, 12, 13, 14, 15, 16, 17, 18, 19, and 20 nucleotides.

Chimeric probes need not be composed of naturally occurring nucleotides. Modified nucleotides, unnatural bases and nucleotide and oligonucleotide analogs can be used. All that is required is that the primer has the general structure described herein and be capable of the interactions and reactions required in the disclosed method. The DNA regions of chimeric probes generally can be made up of deoxyribonucleotides. Modified forms of nucleotides can be used so long as a hybrid between the DNA region 5′ of the RNA region and an RNA molecule is recognized and used as a substrate for the DNA/RNA hybrid-specific ribonuclease. Modified phosphate linkages can fall in this category of modification.

The RNA region of chimeric probes can generally be made up of ribonucleotides. Modified forms of nucleotides can be used so long as the RNA region can hybridize to the target RNA sequence and so long as a hybrid between the RNA region and an RNA molecule is not recognized and used as a substrate for the DNA/RNA hybrid-specific ribonuclease. Derivatized 2′-hydroxys and modified phosphate linkages can fall in this category of modification. For example, one of more of the nucleotides in the RNA region of the probe can be 2′-O-methyl ribonucleotides.

As used herein, a ribonucleotide is a nucleotide having a 2′ hydroxyl function. Analogously, a 2′-deoxyribonucleotide is a nucleotide having only 2′ hydrogens. Thus, ribonucleotides and deoxyribonucleotides as used herein refer to naturally occurring nucleotides having nucleoside components adenosine, guanosine, cytidine, and uridine, or 2′-deoxyadenosine, 2′-deoxyguanosine, 2′-deoxycytidine, and thymidine, respectively, without any chemical modification.

### B. Probe Arrays

Different chimeric probes can be used together as a set. The set can be used as a mixture of all or subsets of the probes, probes used separately in separate reactions, or immobilized in an array. Probes used separately or as mixtures can be physically separable through, for example, the use of first labels, sorting tags, or immobilization on beads. A probe array (also referred to herein as an array) includes a plurality of probes, such as chimeric probes, immobilized at identified or predetermined locations on the array. In this context, a plurality of probes refers to a multiple probes each having a different sequence. Each predetermined location on the array has one type of probe (that is, all the probes at that location have the same sequence). Each location will have multiple copies of the probe. The spatial separation of probes of different sequence in the array allows separate detection and identification of RNA molecules that hybridize to the probes. If a RNA molecule is detected at a given location in a probe array, it indicates that the sequence adjacent to the site in the nucleic acid fragment where the fragment hybridized is complementary to the probe immobilized at that location in the array.

Solid-state substrates for use in probe array can include any solid material to which oligonucleotides can be coupled, directly or indirectly. This includes materials such as acrylamide, cellulose, nitrocellulose, glass, silicon, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, fibers, woven fibers, shaped polymers, particles and microparticles. Preferred forms for solid-state substrates are silicon chips, glass slides, and microtiter dishes.

Methods for immobilization of oligonucleotides to solid-state substrates are well established. Chimeric probes can be coupled to substrates using established coupling methods. Examples of attachment methods include amino-modified oligonucleotides, which can be attached to, for example, epoxy silane or isothiocyante coated glass; succinylated oligonucleotides, which can be attached to, for example, aminophenyl or aminophenyl-derivatized glass; disulfide modified oligonucleotides, which can be attached to, for example, mercaptosilanized glass; and hydrazine, which can be attached to, for example, aldehyde or epoxide. Examples suitable attachment methods are described by Integrated DNA Technologies, “Strategies for Attaching Oligonucleotides to Solid Supports” (2011); Guo et al., Nucleic Acids Res. 22:5456-5465 (1994); Pease et al., Proc. Natl. Acad. Sci. USA 91(11):5022-5026 (1994); Khrapko et al., Mol Biol (Mosk) (USSR) 25:718-730 (1991); Stimpson et al., Proc. Natl. Acad. Sci. USA 92:6379-6383 (1995); U.S. Pat. No. 5,871,928 to Fodor et al.; U.S. Pat. No. 5,654,413 to Brenner; U.S. Pat. No. 5,429,807; and U.S. Pat. No. 5,599,695 to Pease et al.

Although preferred, it is not required that a given probe array be a single unit or structure. The set of probes may be distributed over any number of solid supports. For example, at one extreme, each probe may be immobilized in a separate reaction tube or container. Sorting tags, which are compounds that can be used to sort or separate compounds or complexes having the sorting tag from those that do not, can be used to sort and separate different groups of the parts of a multipart probe array.

The probes in arrays can also be designed to have similar hybrid stability. This would make hybridization of RNA molecules to chimeric probes more efficient and reduce the incidence of mismatch hybridization. The hybrid stability of probes can be calculated using known formulas and principles of thermodynamics (see, for example, Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). The hybrid stability of the probes can be made more similar (a process that can be referred to as smoothing the hybrid stabilities) by, for example, chemically modifying the probes (Nguyen et al., Nucleic Acids Res. 25(15):3059-3065 (1997); Hohsisel, Nucleic Acids Res. 24(3):430-432 (1996)). Hybrid stability can also be smoothed by carrying out the hybridization under specialized conditions (Nguyen et al., Nucleic Acids Res. 27(6):1492-1498 (1999); Wood et al., Proc. Natl. Acad. Sci. USA 82(6):1585-1588 (1985)).

Another way of smoothing hybrid stability of the probes is to vary the length of the probes. This would allow adjustment of the hybrid stability of each probe so that all of the probes had similar hybrid stabilities (to the extent possible). Since the addition or deletion of a single nucleotide from a probe will change the hybrid stability of the probe by a fixed increment, it is understood that the hybrid stabilities of the probes in a probe array will not be equal. For this reason, similarity of hybrid stability as used herein refers to any increase in the similarity of the hybrid stabilities of the probes (or, put another way, any reduction in the differences in hybrid stabilities of the probes). This is useful since any such increased similarity in hybrid stability can improve the efficiency and fidelity of hybridization and coupling of the chimeric probes.

The efficiency of hybridization and coupling of chimeric probes to sample fragments can also be improved by grouping chimeric probes of similar hybrid stability in sections or segments of a probe array that can be subjected to different hybridization conditions. In this way, the hybridization conditions can be optimized for particular classes of probes.

### C. Samples

The disclosed methods can be used to detect and analyze any RNA molecules form any source. Thus, the source of the RNA molecules for the disclosed methods, and of the samples containing RNA molecules, is broad. Samples for use in the disclosed methods generally can be any sample that contains, or may contain, RNA molecules. Any sample from any source can be used with the disclosed method. Examples of suitable target samples include cell samples, tissue samples, cell extracts, components or fractions purified from another sample, environmental samples, culture samples, tissue samples, bodily fluids, and biopsy samples. Numerous other sources of samples are known or can be developed and any can be used with the disclosed method. Preferred samples for use with the disclosed method are samples of cells and tissues.

### D. Labeled Nucleotides

The disclosed methods make use of labels (termed “first labels”) that are incorporated into the extended nucleic acid strand. In the methods, this incorporation is accomplished by incorporation of labeled nucleotides during extension of the strand. Labeled nucleotides for incorporation into nucleic acids being polymerized are well known and can be used in the disclosed methods. Any label and any labeled nucleotide (that can be incorporated by the nucleic acid polymerase) can be used. However, the disclosed method generally makes use of first labels that are members of a label pair. Label pairs are pairs of compounds that bind or interact with one another. Biotin and streptavidin and an antibody and its hapten are examples of label pairs. First labels are preferably one member of a label pair (the other member of the label pair should be used as the specific binding molecule in the label conjugate). Labels pairs and first labels can be chosen such that nucleotides labeled or derivatized with the first label can be incorporated into a nucleic acid strand being synthesized by the nucleic acid polymerase. Useful labeled nucleotides include biotinylated nucleotides, digoxigenin-containing nucleotides, dinitrophenol-containing nucleotides, bromodeoxyuridine, and fluorescent nucleotides (such as fluorescein-containing nucleotides).

First labels are molecules or moieties that are incorporated into the extended nucleic acid strand in the disclosed method and to which specific binding molecules can associate. First labels can be any type of molecule or moiety that can serve as a target for specific binding molecule association.

### E. Label Conjugates

Label conjugates are used in the disclosed methods to associate second labels to labeled nucleotides incorporated into extended nucleic acid strands (thus associating the second labels to the RNA molecules that are extended). They serve as bridges between the RNA molecules to be detected and detection conjugates used to increase the signal for detection. Label conjugates are conjugates of a specific binding molecule and a second label. The specific binding molecule in a label conjugate is chosen to associate with or bind to a first label. The second label in a label conjugate is chosen to associate with or bind to a detection conjugate. Label conjugates can have any structure and any additional components that are consistent with the function of the label conjugate in the disclosed methods.

As used herein, a specific binding molecule is a molecule that interacts specifically with a particular molecule or moiety. The molecule or moiety that interacts specifically with a specific binding molecule is referred to herein as a target molecule. Generally, first labels are the target molecule in the context of the disclosed methods. It is to be understood that the term target molecule refers to both separate molecules and to portions of molecules, such as an epitope of a protein, that interacts specifically with a specific binding molecule. Antibodies, either member of a receptor/ligand pair, and other molecules with specific binding affinities are examples of specific binding molecules, useful as the affinity portion of a reporter binding molecule.

Any specific binding molecule can be used in the disclosed label conjugates. However, the disclosed method generally makes use of specific binding molecules that are members of a label pair. Specific binding molecules are preferably one member of a label pair (the other member of the label pair should be used as the first label). Labels pairs and specific binding molecules can be chosen such that nucleotides labeled or derivatized with the corresponding first label can be incorporated into a nucleic acid strand being synthesized by the nucleic acid polymerase. Specific binding molecules are molecules or moieties that are part of label conjugates and to which first labels can associate. Preferred specific binding molecules include streptavidin, digoxigenin-specific antibodies, dinitrophenol-specific antibodies, bromodeoxyuridine-specific antibodies, and fluorescein-specific antibodies.

A specific binding molecule that interacts specifically with a particular target molecule is said to be specific for that target molecule. For example, where the specific binding molecule is an antibody that binds to a particular antigen, the specific binding molecule is said to be specific for that antigen. The antigen is the target molecule. The label conjugate containing the specific binding molecule can also be referred to as being specific for a particular target molecule, such as a particular first label. Specific binding molecules preferably are antibodies, ligands, binding proteins, receptor proteins, haptens, aptamers, carbohydrates, synthetic polyamides, or oligonucleotides.

Antibodies useful as, for example, specific binding molecules, first labels, and second labels, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Any label can be used as a second label. Second labels are molecules or moieties that are part of label conjugates and to which detection conjugates can associate. Second labels can be any type of molecule or moiety that can serve as a target for detection conjugate association. Preferably, the second label also mediates or allows aggregation of detection conjugates on and at the label conjugate. Preferred second labels include gold, gold nanoparticles, and other gold conjugates.

### F. Detection Conjugate

Detection conjugates are used in the disclosed methods to associate detectable signal to hybridized and extended RNA molecules. Detection conjugates associate with or bind to second labels in label conjugates. Detection conjugates can include or generate any signal. Preferably, detection conjugates aggregate on and around second labels, with the effect that multiple detection conjugates are easier to detect. Detection conjugates can also include multiple copies of labels such that association of one detection conjugate associates many labels with a label conjugate.

Aggregation can be accomplished in any suitable manner. For example, the second label can be chosen such that multiple detection conjugates can associate with or bind to the second label. Association of detection conjugates can be via, for example, binding, covalent coupling, or chemical reaction. Alternatively, or in addition, the detection conjugate can self-aggregate. Aggregation of the detection conjugate can be via an aggregator. An aggregator is a compound multiple copies of which can bind to or associate with a single label conjugate.

A useful detection conjugate is a silver nanoparticle. When used with gold nanoparticles as the second label, silver nanoparticles react with and aggregate on the gold nanoparticle. For example, silver can be reduced in the presence of gold, where the reduction causes silver to build up on the gold surface.

### G. Labels

Labels can be used to produce signal for detection. A label is any molecule that can be included or incorporated in extended nucleic acids strands, label conjugate, or detection conjugates, directly or indirectly, and which results in a measurable, detectable signal, either directly or indirectly. A label is associated with a component when it is coupled or bound, either covalently or non-covalently, to the component. A label is coupled to a component when it is covalently coupled to the component. Many suitable labels for incorporation into, coupling to, or association with nucleic acid are known. Examples of labels suitable for use in the disclosed method are radioactive isotopes, fluorescent molecules, phosphorescent molecules, bioluminescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for simultaneous detection are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are preferred form of label since they can be directly incorporated into nucleic acids during synthesis. Examples of labels that can be incorporated into DNA or RNA include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxigenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labeling. Cy3.5 and Cy7 are available as avidin or anti-digoxigenin conjugates for secondary detection of biotin- or digoxigenin-labeled probes.

Labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.1.1^{3.7}]decane]-4-yl)phenyl phosphate; Tropix, Inc.).

Other labels include molecular or metal barcodes, mass labels, and labels detectable by nuclear magnetic resonance, electron paramagnetic resonance, surface enhanced raman scattering, surface plasmon resonance, fluorescence, phosphorescence, chemiluminescence, resonance raman, microwave, or a combination. Mass labels are compounds or moieties that have, or which give the labeled component, a distinctive mass signature in mass spectroscopy. Mass labels are useful when mass spectroscopy is used for detection. Preferred mass labels are peptide nucleic acids and carbohydrates. Combinations of labels can also be useful. For example, color-encoded microbeads having, for example, 265 unique combinations of labels, are useful for distinguishing numerous components. For example, 256 different chimeric probes can be uniquely labeled and detected allowing multiplexing and automation of the disclosed method.

Useful labels are described in de Haas et al., “Platinum porphyrins as phosphorescent label for time-resolved microscopy,” J. Histochem. Cytochem. 45(9):1279-92 (1997); Karger and Gesteland, “Digital chemiluminescence imaging of DNA sequencing blots using a charge-coupled device camera,” Nucleic Acids Res. 20(24):6657-65 (1992); Keyes et al., “Overall and internal dynamics of DNA as monitored by five-atom-tethered spin labels,” Biophys. J. 72(1):282-90 (1997); Kirschstein et al., “Detection of the DeltaF508 mutation in the CFTR gene by means of time-resolved fluorescence methods,” Bioelectrochem. Bioenerg. 48(2):415-21 (1999); Kricka, “Selected strategies for improving sensitivity and reliability of immunoassays,” Clin. Chem. 40(3):347-57 (1994); Kricka, “Chemiluminescent and bioluminescent techniques,” Clin. Chem. 37(9):1472-81 (1991); Kumke et al., “Temperature and quenching studies of fluorescence polarization detection of DNA hybridization,” Anal. Chem. 69(3):500-6 (1997); McCreery, “Digoxigenin labeling,” Mol. Biotechnol. 7(2):121-4 (1997); Mansfield, et al., “Nucleic acid detection using non-radioactive labeling methods,” Mol. Cell Probes 9(3):145-56 (1995); Nurmi, et al., “A new label technology for the detection of specific polymerase chain reaction products in a closed tube,” Nucleic Acids Res. 28(8):28 (2000); Oetting et al. “Multiplexed short tandem repeat polymorphisms of the Weber 8A set of markers using tailed primers and infrared fluorescence detection,” Electrophoresis 19(18):3079-83(1998); Roda et al., “Chemiluminescent imaging of enzyme-labeled probes using an optical microscope-videocamera luminograph,” Anal. Biochem. 257(1):53-62 (1998); Siddiqi et al., “Evaluation of electrochemiluminescence- and bioluminescence-based assays for quantitating specific DNA,” J. Clin. Lab. Anal. 10(6):423-31 (1996); Stevenson et al., “Synchronous luminescence: a new detection technique for multiple fluorescent probes used for DNA sequencing,” Biotechniques 16(6):1104-11 (1994); Vo-Dinh et al., “Surface-enhanced Raman gene probes,” Anal. Chem. 66(20):3379-83 (1994); Volkers et al., “Microwave label detection technique for DNA in situ hybridization,” Eur. J. Morphol. 29(1):59-62 (1991).

Metal barcodes, a form of molecular barcode, are 30-300 nm diameter by 400-4000 nm multilayer multi metal rods. These rods are constructed by electrodeposition into an alumina mold, then the alumina is removed leaving these small multilayer objects behind. The system can have up to 12 zones encoded, in up to 7 different metals, where the metals have different reflectivity and thus appear lighter or darker in an optical microscope depending on the metal; this leads to practically unlimited identification codes. The metal bars can be coated with glass or other material, and probes attached to the glass using methods commonly known in the art; assay readout is by fluorescence from the target, and the identity of the probe is from the light dark pattern of the barcode.

Methods for detecting and measuring signals generated by labels are known. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. Such methods can be used directly in the disclosed method of amplification and detection. As used herein, detection molecules are molecules which interact with amplified nucleic acid and to which one or more detection labels are coupled. In another form of detection, labels can be distinguished temporally via different fluorescent, phosphorescent, or chemiluminescent emission lifetimes. Multiplexed time-dependent detection is described in Squire et al., J. Microscopy 197(2):136-149 (2000), and WO 00/08443.

Quantitative measurement of the amount or intensity of a label can be used. For example, quantitation can be used to determine if a given label, and thus the labeled component, is present at a threshold level or amount. A threshold level or amount is any desired level or amount of signal and can be chosen to suit the needs of the particular form of the method being performed.

### H. Ribonucleases

The disclosed methods use a ribonuclease specific for RNA/DNA hybrids to degrade the portion of an RNA molecule that is hybridized to the DNA region(s) of a chimeric probe. Any ribonuclease that depends on an RNA/DNA hybrid can be used in the disclosed method. The preferred ribonuclease is the well-characterized RNase H (EC 3.1.26.4). Optimal activity of RNase H is achieved at a pH comprised between 7.5 and 9.1 (Berkower et al., J Biol Chem 248:5914-5921 (1973)) in the presence of reducing reagents. RNase H activity is inhibited in the presence of N-ethylmaleimide (a chemical that react with SH groups), and is not markedly affected by high ionic strength (50% activity is retained in the presence of 0.3 M NaCl. RNase H requires Mg²⁺ ions, which can be replaced partially by Mn²⁺ ions.

### I. Nucleic Acid Polymerases

The disclosed methods us a nucleic acid polymerase to extend the end of RNA molecules hybridized to chimeric primers. Any suitable nucleic acid polymerase can be used that is capable of extending a RNA strand using a DNA template and is capable of incorporating the labeled nucleotides in the extension from the RNA strand. Most DNA polymerases satisfy these requirements. Preferred DNA polymerases lack 5′ to 3′ exonuclease activity. Examples of useful nucleic acid polymerases include Klenow fragment (large fragment of DNA polymerase I) (Jacobsen et al., Eur. J. Biochem. 45:623-627 (1974)), T4 DNA polymerase (Kaboord and Benkovic, Curr. Biol. 5:149-157 (1995)), and modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)). Other useful nucleic acid polymerases include bacteriophage φ29 DNA polymerase (U.S. Pat. Nos. 5,198,543 and 5,001,050 to Blanco et al.), phage M2 DNA polymerase (Matsumoto et al., Gene 84:247 (1989)), phage φPRD1 DNA polymerase (Jung et al., Proc. Natl. Acad. Sci. USA 84:8287 (1987)), VENT® DNA polymerase (Kong et al., J. Biol. Chem. 268:1965-1975 (1993)), T5 DNA polymerase (Chatterjee et al., Gene 97:13-19 (1991)), PRD1 DNA polymerase (Zhu and Ito, Biochim. Biophys. Acta. 1219:267-276 (1994)), T7 DNA polymerase (Studier et al., Methods Enzymol. 185:60-89 (1990)), DEEP VENT® DNA polymerase (New England Biolabs, Beverly, Mass.), Thermus flavus DNA polymerase (MBR, Milwaukee, Wis.), and the Stoffel fragment of Taq DNA polymerase (Lawyer et al., PCR Methods Appl. 2(4):275-287 (1993), King et al., J. Biol. Chem. 269(18):13061-13064 (1994)).

### J. Stabilized Nucleic Acids

Although unmodified oligoribonucleotides can function as effective probes, chimeric probes and probe arrays can be more stable and have longer shelf lives if the probes are stabilized by using nucleic acid modifications. Chemical modifications can be made which greatly enhance the nuclease resistance of chimeric probes. In general, such modifications can be made at the 2′ position of the nucleotides in a chimeric probe, and in the phosphate linkages between the nucleotides in a chimeric probe. For example, one or more of the bases of chimeric probe can be 2′ methoxy ribonucleotides, phosphorothioate deoxyribonucleotides, or phosphorothioate ribonucleotides using available nucleic acid synthesis methods. Modified nucleotides and oligonucleotides, and methods for their synthesis, are known. Some of these are described in Offensperger et al., EMBO J., 12:1257-1262 (1993); WO 93/01286 by Rosenberg et al.; Agrawal et al., Proc. Natl. Acad. Sci. USA, 85:7079-7083 (1988); Sarin et al., Proc. Natl. Acad. Sci. USA, 85:7448-7794 (1989); Shaw et al., Nucleic Acids Res, 19:747-750 (1991); Orson et al., Nucl. Acids Res., 19:3435-3441 (1991); Paolella et al., EMBO J., 11:1913-1919 (1992); Pieken, et al., Science, 253:314-317 (1991); Heidenreich and Eckstain, J. Biol. Chem, 267:1904-1909 (1992); WO 91/17093 by Hybridon, Inc.; EP 0339842 by Ajinomoto Co., Inc.; WO 95/23225 by Ribozyme Pharmaceuticals, Inc.; WO 94/15619 by Johns Hopkins University; and U.S. Pat. No. 5,334,711 to Sproat et al.

In describing substituents used to modify nucleotides, oligonucleotides, and chimeric probes, alkyl or alkyl group refers to a saturated aliphatic hydrocarbon, including straight chain, branch chain, and cyclic alkyl groups. For this use it is preferred that such alkyl groups have 1 to 12 carbons. It is more preferred that such alkyl groups have 1 to 6 carbons. It is still more preferred that such alkyl groups have 1 to 2 carbons. It is most preferred that such alkyl groups have 1 carbon. These alkyl groups can also include one or more hydroxyl groups, one or more amino groups, or both. Such hydroxyl and amino groups can be coupled to any carbon atom in the alkyl group. As used herein, the term hydroxy alkyl is used to refer to an alkyl group including one or more hydroxyl groups, the term amino alkyl is used to refer to an alkyl group including one or more amino groups, and hydroxylamino alkyl is used to refer to an alkyl group including one or more hydroxyl groups and one or more amino groups. As used herein, allyl or allyl group refers to an unsaturated aliphatic hydrocarbon, including straight chain, branch chain, and cyclic allyl groups. For this use it is preferred that such allyl groups have 1 to 12 carbons. It is more preferred that such allyl groups have 1 to 6 carbons. It is still more preferred that such allyl groups have 2 to 3 carbons. It is most preferred that such allyl groups have 3 carbons. Other substituents can also be used to modify the nucleotides, oligonucleotides and chimeric probes described herein, such as aryl, alkaryl, and arylalkyl, where aryl refers to a benzyl group, alkaryl refers to an alkyl group substituted with an aryl group, and arylalkyl refers to an aryl group substituted with an alkyl group.

Use herein of the term modification in reference to nucleotides, oligonucleotides, and chimeric probes is intended to refer to chemical differences of a nucleotide or oligonucleotide relative to conventional nucleotides and oligonucleotides. Use of the term modification herein is not intended to limit the manner in which the modified nucleotides, oligonucleotides, and chimeric probes are produced. Similarly, references to replacing a chemical group on a nucleotide, oligonucleotide, or chimeric probe is intended to refer to chemical differences of a nucleotide or oligonucleotide relative to conventional nucleotides and oligonucleotides, and is not intended to limit the manner in which the nucleotides, oligonucleotides, or chimeric probes are produced.

Modifications at the 3′ and 5′ ends: It is well documented that degradation of oligonucleotide analogues is mainly attributable to 3′-exonucleases. Several studies have also demonstrated that various 3′-modifications can greatly decrease the nuclease susceptibility of these analogues. Thus, another method to reduce susceptibility to 3′ exonucleases is introduction of a free amine to a 3′ terminal hydroxyl group of the chimeric probe (see, for example, Orson et al., Nucl. Acids Res., 19:3435-3441 (1991)). Another useful 3′ terminal modification is to couple a thymine nucleotide end of a chimeric probe with a 3′ to 3′ linkage. Such a structure is referred to herein as 3′-3′-thymine nucleotide or T(3′-3′).

Preferred 3′ modifications are those where the 3′ hydroxyl of the chimeric probe is replaced with a chemical group such as —H, —O—R¹, —NH₂, —NH—R¹, —N R¹₂, F, and -3′-nucleotide, where each R¹ is independently alkyl, hydroxy alkyl, amino alkyl, hydroxylamino alkyl, allyl, —PR²(O)—R², or —PR²(S)—R², where each R² is independently O, S, F, alkyl, hydroxy alkyl, amino alkyl, hydroxylamino alkyl, allyl, O—R³, or S—R³, and where each R³ is independently alkyl, hydroxy alkyl, amino alkyl, hydroxylamino alkyl, or allyl. As used herein, the 3′ hydroxyl of a chimeric probe refers to the hydroxyl group that would normally be present on the 3′ carbon of the ribose residue in the 3′ terminal nucleotide of the chimeric probe. As used herein, the 3′ carbon of a chimeric probe refers to the 3′ carbon of the ribose residue in the 3′ terminal nucleotide of the chimeric probe.

Although it is preferred that the 5′ end of chimeric probe have a hydroxyl or phosphate group, the 5′ end can be modified to increase resistance of the chimeric probe to nucleases. Preferred 5′ modifications are those where the 5′ hydroxyl of the chimeric probe is replaced with a chemical group such as —H, —O—R⁴, —NH₂, —NH—R⁴, —N R⁴₂, and F, where each R⁴ is independently alkyl, hydroxy alkyl, amino alkyl, hydroxylamino alkyl, allyl, —PR⁵(O)—R⁵, or —PR⁵(S)—R⁵, where each R⁵ is independently O, S, F, alkyl, hydroxy alkyl, amino alkyl, hydroxylamino alkyl, allyl, O—R⁶, or S—R⁶, and where each R⁶ is independently alkyl, hydroxy alkyl, amino alkyl, hydroxylamino alkyl, or allyl. As used herein, the 5′ hydroxyl of a chimeric probe refers to the hydroxyl that would normally be present on the 5′ carbon of the ribose residue in the 5′ terminal nucleotide of the chimeric probe to which a phosphate group would normally be attached. As used herein, the 5′ carbon of a chimeric probe refers to the 5′ carbon of the ribose residue in the 5′ terminal nucleotide of the chimeric probe. Another useful modification is covalent attachment of an intercalating agent, such as an acridine derivative, to the 5′ terminal phosphate (for example, using a pentamethylene bridge) (see, for example, Maher et al., Science, 245:725-730 (1989); Grigoriev et al., J. Biol. Chem., 267:3389-3395 (1992)).

Modifications at the 2′ position of nucleotides: Another useful class of chemical modifications is modification of the 2′ OH group of a nucleotide's ribose moiety, which has been shown to be critical for the activity of the various intracellular and extracellular nucleases. Typical 2′ modifications are the synthesis of 2′-O-methyl oligonucleotides (Paolella et al., EMBO J., 11:1913-1919 (1992)) and 2′-fluoro and 2′-amino-oligonucleotides (Pieken, et al., Science, 253:314-317 (1991); Heidenreich and Eckstain, J. Biol. Chem, 267:1904-1909 (1992)). Chimeric probes also contain deoxyribonucleotides (in the DNA region(s)). However, to the extent that the ribonuclease used requires an unsubstituted hydrogen at the 2′ position to recognize RNA/DNA hybrids, 2′ modification should not be used in the nucleotides of the DNA region of chimeric probes that is 5′ of the RNA region.

Preferred 2′ modifications are those where the 2′ hydroxyl of a nucleotide is replaced with a chemical group such as —H, —O—R⁷, —NH₂, —NH—R⁷, —N R⁷₂, F, and -2′-nucleotide, where each R⁷ is independently alkyl, hydroxy alkyl, amino alkyl, hydroxylamino alkyl, allyl, —PR⁸(O)—R⁸, or —PR⁸(S)—R⁸, where each R⁸ is independently O, S, F, alkyl, hydroxy alkyl, amino alkyl, hydroxylamino alkyl, allyl, O—R⁹, or S—R⁹, and where each R⁹ is independently alkyl, hydroxy alkyl, amino alkyl, hydroxylamino alkyl, or allyl. More preferred 2′ modifications are those where the 2′ hydroxyl of a nucleotide is replaced with a chemical group such as —H, —O—CH₃, —NH₂, —NH—CH₃, —N—(CH₃)₂, F, —OCH₂—CH═CH₂, —OPO(O)—CH₃, and —OPO(S)—CH₃. The most preferred 2′ modification is where the 2′ hydroxyl of a nucleotide is replaced with —O—CH₃.

Modifications to the phosphate linkages: Modification to the phosphate groups linking nucleotides in a chimeric probe can also be used to enhance the resistance of the chimeric probe to nucleases. Typical modifications for this purpose include replacing one or both of the free oxygen atoms sulfur or a halogen. The free oxygen atoms, or a sulfur atom, if present, can also be linked to chemical groups such as alkyl, hydroxy alkyl, amino alkyl, hydroxylamino alkyl, or allyl. Examples of such substitutions, such as the use of 3′ and/or 5′ dihalophosphonate substituted nucleotides (for example, 3′ and/or 5′-CF₂-phosphonate substituted nucleotides), are described in WO 95/23225. Preferred modified phosphate linking groups for use in chimeric probes include —OPR¹⁰(O)O—, —OPR¹⁰(S)O—, and —OPO(S)O—, where R¹⁰ is alkyl, hydroxy alkyl, amino alkyl, hydroxylamino alkyl, allyl, —O—R¹¹, —NH₂, —NH—R¹¹, —N R¹¹₂, or F, and where each R¹¹ is independently alkyl, hydroxy alkyl, amino alkyl, hydroxylamino alkyl, or allyl. More preferred modified phosphate linking groups for use in chimeric probes include —OPR¹²(O)O—, —OPR¹²(S)O—, and —OPO(S)O—, where R¹² is —CH₃, —O—CH₃, —OCH₂—CH═CH₂, —NH₂, —NH—CH₃, —N—(CH₃)₂, or F. The most preferred modified phosphate linking group for use in chimeric probes is —OPO(S)O—, which is commonly referred to as a phosphorothioate.

Another useful modification is methylation of cytosine bases that may be present in the sequence. The stability of chimeric probe/RNA molecule hybrids can be increased by using modified nucleotides that result in oligonucleotides with stronger base pairing to the RNA molecules. For example, C-5 propynyl pyrimide nucleotides increase hydrogen bonding between nucleic acids (Froehler et al., Tetrahedron Letters 33:5307-5310 (1992)).

The above modifications can be used in limited regions of the chimeric probes and/or in combinations to result in chimeras of modified chimeric probes. Certain regions of chimeric probes are more amenable to modification than others due to the requirement for recognition of the RNA/DNA hybrid region by the ribonuclease and the DNA region by the nucleic acid polymerase. For example, it has been discovered that 2′-O-methyl nucleotides can be introduced into the RNA region of chimeric probes without affecting the disclosed methods.

### K. Oligonucleotide Synthesis

Chimeric probes and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

### L. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting RNA molecules, the kit comprising a probe array, labeled nucleotides and a label conjugate. The kits also can contain detection conjugates. The disclosed kits can also include a ribonuclease, a nucleic acid polymerase, or both.

### M. Mixtures

Disclosed are mixtures formed by performing or preparing to perform the disclosed method. For example, disclosed are mixtures comprising a probe array and a sample; a probe array and an RNA molecule; a probe array and a ribonuclease; a probe array and labeled nucleotides; a probe array and a nucleic acid polymerase; a probe array and a label conjugate; a probe array and a detection conjugate; a probe array, an RNA molecule, and a ribonuclease; a probe array, an RNA molecule, and a nucleic acid polymerase; a probe array, an RNA molecule, labeled nucleotides, and a nucleic acid polymerase; a probe array, an RNA molecule, a ribonuclease, and a nucleic acid polymerase; and a probe array, an RNA molecule, a ribonuclease, labeled nucleotides, and a nucleic acid polymerase.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### N. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising a probe array and a reaction controller (e.g., PCR machine, automatic pipetting system and reaction incubator).

### O. Data Structures and Computer Control

Disclosed are data structures used in, generated by, or generated from, the disclosed method. Data structures generally are any form of data, information, and/or objects collected, organized, stored, and/or embodied in a composition or medium. RNA detection results stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, RNA detection and analysis. In particular, the disclosed methods and compositions can be used to detect particular RNA molecules of interest, such as RNA molecules characteristic of pathogens, disease cells, and diseases. Other uses include generating expression catalogs for cells, samples, etc. Other uses are disclosed, apparent from the disclosure, and/or will be understood by those in the art.

### A. Actions Based on Identifications

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, subjects, locations, or environments can be identified as having particular microorganisms based on RNA molecules detected. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising making one or more further identifications based on one or more other identifications. For example, particular treatments, monitorings, follow-ups, advice, etc. can be identified based on the other identification. For example, identification of a subject as having a disease or condition with a high level of a particular component or characteristic can be further identified as a subject that could or should be treated with a therapy based on or directed to the high level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods comprising treating, monitoring, following-up with, advising, etc. a subject identified in any of the disclosed methods. Also disclosed are methods comprising treating, monitoring, following-up with, advising, etc. a subject for which a record of an identification from any of the disclosed methods has been made. For example, particular treatments, monitorings, follow-ups, advice, etc. can be used based on an identification and/or based on a record of an identification. For example, a subject identified as having a disease or condition with a high level of a particular component or characteristic (and/or a subject for which a record has been made of such an identification) can be treated with a therapy based on or directed to the high level component or characteristic. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

### Methods

Disclosed are methods for detecting RNA in a sample. In some forms, the method involves (a) bringing into contact the sample and a probe array, (b) bringing into contact the probe array and a ribonuclease specific for RNA/DNA hybrids (such as RNase H), (c) bringing into contact the probe array, labeled nucleotides, and a nucleic acid polymerase capable of extending a RNA strand using a DNA template and capable of incorporating the labeled nucleotides in the extension from the RNA strand (such as Klenow fragment DNA polymerase), and (d) detecting the labeled nucleotides in the extended nucleic acid strand. The probe array comprises one or more chimeric probes. The chimeric probes comprise a DNA region and a RNA region, where the DNA region and the RNA region are contiguous and where the DNA region is 5′ of the RNA region. The chimeric probe can also include a second DNA region. The second DNA region can also be contiguous with the RNA region and can be 3′ of the RNA region. If the sample contains an RNA molecule complementary to a nucleotide sequence of at least one of the chimeric probes, the RNA molecule will hybridize with the complementary chimeric probe. In some forms, bringing into contact the sample and a probe array, bringing into contact the probe array and the ribonuclease, and bringing into contact the probe array, labeled nucleotides, and the nucleic acid polymerase (steps (a), (b), and (c)) are carried out simultaneously.

In some forms of the method, labeling and detection can be enhanced by labeling the first label. This can be accomplished, for example, by bringing into contact the probe array and a label conjugate, where the label conjugate comprises a specific binding molecule and a second label, and where the specific binding molecule binds to the first label. The labeled nucleotides in the extended nucleic acid strand are then detected by detecting the second label. An example of a useful combination of first label and label conjugate are biotin as the first label and a streptavidin-conjugated gold nanoparticle as the label conjugate. In this example, the streptavidin is the specific binding molecule that binds to the first label (biotin) and gold nanoparticle is the second label.

By using another conjugate that can aggregate on or at the site of the second label, the amount of second label at the site is increased, increasing the sensitivity of the detection and making detection easier. This can be accomplished, for example, by bringing into contact the probe array and a detection conjugate, where detection conjugate comprises an aggregator, and where the aggregator mediates aggregation of detection conjugates on the label conjugate. An example of a useful combination of second label and detection conjugate are gold nanoparticle as the second label and a silver nanoparticle as the detection conjugate. In this example, the silver nanoparticle is the aggregator. The silver nanoparticle reacts with the gold nanoparticle to accumulate silver nanoparticles at the site of the gold nanoparticles. The accumulation of the reacted silver nanoparticles can be sufficient to detect with the naked eye.

Rapid and accurate detection of pathogens, such as RNA viruses at the point of care, will allow proper patient treatment and save lives. The disclosed RNA microchip can directly detect RNA without reverse transcription and PCR amplification. The disclosed RNA microchip can use nanoparticle-assisted signal amplification. The disclosed RNA microchip technology is simple and accurate and can differentiate single-nucleotide difference. In some forms, RNA can be sensitively detected within 1 hour, and the signal can be detected by naked eye. The visual readout format and simplicity make the disclosed RNA microchip technology well suited for the rapid and accurate detection of pathogens in clinics and in the field with minimum resources.

The disclosed technology can also be used to detect and analyze any RNA or combination of RNA from single or multiple sources. For example, the technology can be used to detect RNA expression patterns in cells and samples. As another example, the probe array can include one or more of the chimeric probes that are complementary to a nucleotide sequence characteristic of, for example, a virus, a bacteria, or a microbe. Detection of the characteristic nucleotide sequence indicates the presence of the corresponding virus, bacteria, or microbe.

### A. Sample Preparation

Samples can be prepared using any suitable technique. Generally, all that is required is the removal or reduction of interfering components in the sample (such as nucleases), if any, and prevention of destruction of the RNA molecules. It is noted, however, that purification of the RNA is not required. Simple preparation techniques are sufficient for use in the disclosed methods. Because small fragments of RNA molecules are sufficient and preferred for use in the disclosed methods, some degradation of RNA in the sample is preferred. The preferred preparation of RNA from cell samples is NaOH treatment without any RNA isolation. The RNA sample can be prepared by treating a biological specimen with NaOH for a few minutes at an elevated temperature, which hydrolyzes biological RNAs into short fragments for rapid hybridization and detection. After neutralization and centrifugation, the RNA sample (supernate) is ready for the direct detection of the RNA.

As an example, a sample can be prepared in less than 10 min by treating the sample (such as bacteria or isolated bacteria total RNA) with NaOH (such as 200 mM) for 5 minutes at an elevated temperature, followed by neutralization and centrifugation. The NaOH treatment fragments biological RNA (such as mRNA, normally several thousand nucleotides in length) into short pieces (such as 15-25 nucleotides), which facilitates hybridization of the RNA molecules to the probe array (Spencer et al., ChemBioChem, 11, 1378 (2010)). The sample can also be prepared using any suitable RNA isolation or preparation technique.

### B. Hybridization

In the method, RNA molecules in the sample can hybridize to chimeric probes having complementary sequence. Hybridization can be accomplished under a variety of conditions. Generally, the temperature should not be above the melting temperature of a hybrid between the RNA molecule and the RNA region of the chimeric probe. The pH and salt conditions can vary, but neutral pH and moderate salt conditions are preferred. Most preferred are pH, salt, and temperatures that are compatible with the ribonuclease and the nucleic acid polymerase. Such conditions are well known or their determination is within the knowledge of those in the field of nucleic acid reactions.

### C. RNA Degradation

The portion of an RNA molecule hybridized to the DNA region of the chimeric probe is degraded by the ribonuclease specific for RNA/DNA hybrids. This degradation can be carried out under any suitable conditions. Generally, the temperature should not be above the melting temperature of a hybrid between the RNA molecule and the RNA region of the chimeric probe. The pH and salt conditions can vary, but neutral pH and moderate salt conditions are preferred. Most preferred are pH, salt, and temperatures that are compatible with the ribonuclease and the nucleic acid polymerase.

### D. Extension of RNA Molecule

The hybridized RNA molecule can then extended to form an extended nucleic acid strand, where the DNA region of the chimeric probe that is 5′ of the RNA region serves as the template. For labeling, at least one labeled nucleotide is incorporated into the extended nucleic acid strand. The labeled nucleotides comprise a first label. Because of the sequence relationship between the chimeric probe and the RNA molecule that hybridizes to the chimeric probe, detection of the labeled nucleotides in the extended nucleic acid strand indicates the presence of the RNA molecule in the sample.

This extension can be carried out under any suitable conditions. Generally, the temperature should not be above the melting temperature of a hybrid between the RNA molecule and the RNA region of the chimeric probe. The pH and salt conditions can vary, but neutral pH and moderate salt conditions are preferred. Most preferred are pH, salt, and temperatures that are compatible with the ribonuclease and the nucleic acid polymerase.

Because the conditions for hybridization of the RNA molecule, for degradation of the RNA in the RNA/DNA hybrid, and for extension of the RNA molecule can be compatible, these operations and reactions can be carried out simultaneously.

### E. Secondary Labeling

In some forms of the method, labeling and detection can be enhanced by labeling the first label. This can be accomplished, for example, by bringing into contact the probe array and a label conjugate, where the label conjugate comprises a specific binding molecule and a second label, and where the specific binding molecule binds to the first label. The labeled nucleotides in the extended nucleic acid strand are then detected by detecting the second label. An example of a useful combination of first label and label conjugate are biotin as the first label and a streptavidin-conjugated gold nanoparticle as the label conjugate. In this example, the streptavidin is the specific binding molecule that binds to the first label (biotin) and gold nanoparticle is the second label.

### F. Aggregation (Tertiary Labeling)

By using another conjugate that can aggregate on or at the site of the second label, the amount of second label at the site is increased, increasing the sensitivity of the detection and making detection easier. This can be accomplished, for example, by bringing into contact the probe array and a detection conjugate, where detection conjugate comprises an aggregator, and where the aggregator mediates aggregation of detection conjugates on the label conjugate. An example of a useful combination of second label and detection conjugate are gold nanoparticle as the second label and a silver nanoparticle as the detection conjugate. In this example, the silver nanoparticle is the aggregator. The silver nanoparticle reacts with the gold nanoparticle to accumulate silver nanoparticles at the site of the gold nanoparticles. The accumulation and aggregation of the formed silver nanoparticles can be sufficient to detect with the naked eye (or with assistance of a magnifying glass).

### G. Detection

The disclosed methods allow sensitive detection of RNA molecules by adding labeled components where RNA molecules have hybridized in a probe array. In various forms of the disclosed methods, RNA molecules can be detected by, for example, (a) detecting labeled nucleotides (via the first labels) incorporated into extended strands of the RNA molecules, (b) detecting second labels in label conjugates associated with the RNA molecules, (c) detecting detection conjugates associated or aggregated with the RNA molecules, or a combination. Preferably, RNA molecules can be detected by, for example detecting detection conjugates associated or aggregated with the RNA molecules via label conjugates associated with the RNA molecules.

The component being detected can be detected using any technique or means that allows identification and discrimination of the component to be detected from other components and materials. Generally, components and labels to be detected can be matched to appropriate detection techniques. For example, components and labels that produce a fluorescent signal can be detected with, for example, a spectrophotomer or by photography. Components and labels that produce a visible light signal can be detected, for example, visually, with a spectrophotometer, or by photography. Components and labels that produce radioactivity can be detected, for example, by scintillation counting or autoradiography. Visual detection is preferred.

The disclosed labels and components can also be detected with established enzyme-linked detection systems. For example, amplified nucleic acid labeled by incorporation of biotin-16-UTP (Boehringher Mannheim) can be detected as follows. The nucleic acid is immobilized on a solid glass surface by hybridization with a complementary DNA oligonucleotide (address probe) complementary to the target sequence (or its complement) present in the amplified nucleic acid. After hybridization, the glass slide is washed and contacted with alkaline phosphatase-streptavidin conjugate (Tropix, Inc., Bedford, Mass.). This enzyme-streptavidin conjugate binds to the biotin moieties on the amplified nucleic acid. The slide is again washed to remove excess enzyme conjugate and the chemiluminescent substrate CSPD (Tropix, Inc.) is added and covered with a glass cover slip. The slide can then be imaged in a Biorad Fluorimager.

### H. Discrimination Between Closely Related RNA Molecules

Chimeric probes can be designed to discriminate closely related target sequences, such as genetic alleles. As shown in the examples, the disclosed method can accurately identify an RNA molecule from RNA molecules differing by a single nucleotide. This is best accomplished by including a differentiating nucleotide(s) in the DNA region of the chimeric probe that is 5′ of the RNA region. Because the hybridization, RNase H digestion, and DNA polymerase extension are dependent on the base-pairing, and because base-pairing in the DNA region of the chimeric probe that is 5′ of the RNA region involves all of these steps, differentiation is enhanced. These three layers of discrimination can help straightforwardly achieving high detection specificity. The combination of these discriminative forces easily offers single-nucleotide discrimination.

### I. Detecting Groups of RNA Molecules

Multiplex RNA detection is particularly useful for detecting mutations in genes where numerous distinct mutations are associated with certain diseases or where mutations in multiple genes are involved. For example, although the gene responsible for Huntington's chorea has been identified, a wide range of mutations in different parts of the gene occur among affected individuals. The result is that no single test has been devised to detect whether an individual has one or more of the many Huntington's mutations. A single RNA detection assay can be used to detect the presence of one or more members of a group of any number of RNA molecules. This can be accomplished, for example, by designing chimeric probes for each RNA molecule in the group. All of the chimeric probes are placed in the same location or spot in the probe array. If any of the RNA molecules are present in the sample, its presence will be detected at the corresponding location in the probe array. Since the first labels, label conjugates, and detection conjugates can be the same for all of the probes and all of the RNA molecules, a signal can be produced in the presence of any or any combination of the RNA molecules in the group. Detection indicates that at least one member of the RNA molecule group is present in the sample.

### J. Treatment

Subjects and objects identified as having specific pathogens can then be treated to reduce or eliminate the pathogen. Numerous compositions and methods are known for such treatments both in general and for specific pathogens. Such treatments can be used with, based on, or following the disclosed methods.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

A cell can be in vitro. Alternatively, a cell can be in vivo and can be found in a subject. A “cell” can be a cell from any organism including, but not limited to, a bacterium.

### EXAMPLES

A. RNA Microchip Detection Using Nanoparticle-Assisted Signal Amplification

This example describes an example of the disclosed RNA microchip used to directly detect RNA via direct visualization and nanoparticle-assisted signal amplification. This technology is simple and accurate and it can efficiently differentiate single-nucleotide difference. The RNA sample preparation is simple and can be accomplished in 5 min. The disclosed rapid RNA detection can be completed within 45 min (including sample preparation time) and have the detection sensitivity at the low fmole level. In addition, the detection signals can be observed by naked eye or with a magnifying glass. This approach is PCR-free, easy to perform and cost-effective. Moreover, the strategy does not require sophisticated equipment and can be used to help monitor disinfection treatment. The visual detection format and simplicity make this RNA microchip technology well suited for rapid and accurate detection of pathogens in remote areas, clinics, and fields with minimum equipment. The disclosed simple, rapid and accurate technology will greatly benefit the point-of-care applications, especially during infectious disease outbreaks.

This example describes an example of the disclosed simple RNA microchip (FIG. 1) for rapid and direct RNA detection via nanoparticle formation and visualization. The nanoparticle assists signal amplification and visual detection by naked eye (seeing objects as small as 40 μm in size) or with assistance of a simple tool (such as a magnifying glass). The RNA detection system is simple: after target RNA hybridization to RNA microchip immobilized with RNA probes (Spencer et al., ChemBioChem, 11, 1378 (2010)), the bound RNAs are digested with RNase H and extended with biotin-labeled dNTPs by Klenow DNA polymerase. Using the conjugate of streptavidin and gold nanoparticle (Au-NP), the biotin labels incorporated to the target RNAs are then converted to Au-NP labels. The Au-NP can catalyze the formation of silver nanoparticles (Ag-NP) via the silver staining (Taton et al., Science, 289, 1757 (2000); Cao et al., Biosens Bioelectron, 22, 393 (2006): Qi et al., Anal Bioanal Chem, 398, 2745 (2010); Tang et al., Diagn Microbiol Infect Dis, 65, 372 (2009); Zhou et al., J Am Chem Soc, 132, 6932 (2010)), allowing the signal amplification. The formed Ag-NPs (FIGS. 1 and 2) aggregate and deposit as dark spots on the RNA probe sites. An image of an array of these dark spots, which are by naked human eye, will form for direct visual observation (FIG. 2). It was also demonstrated that this RNA microchip can differentiate single nucleotide difference and detect RNA at the low fmolar level.

The method is aided by a simple sample preparation protocol for biological RNAs. The RNA sample preparation is convenient: just treating biological samples (such as bacteria) directly with NaOH for 3 minutes. Unlike other technologies, the isolation and purification of RNAs are not necessary. With the disclosed RNA microchip method, simple and rapid RNA detection can be accomplished within 45 minutes.

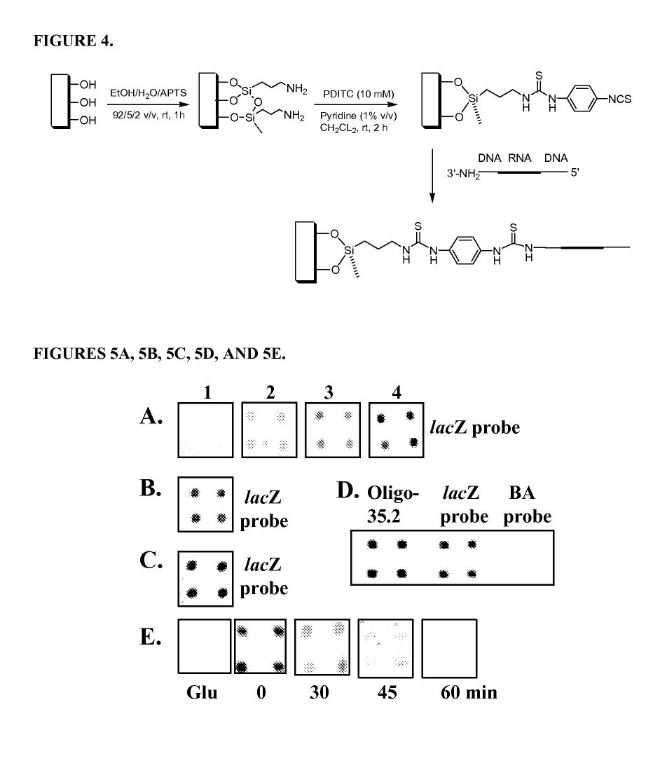

The RNA detection system is based on a RNA 3′-labeling approach, where a DNA polymerase directly incorporates labeled dNTPs into RNA on a DNA template (Huang and Alsaidi, Analytical Biochemistry, 322, 269 (2003); Alsaidi et al., ChemBioChem, 5, 1136 (2004)). In order to directly detect RNA, functionalized RNA probes were designed. A RNA probe consists of the RNA 3′-region and the DNA 5′-region (FIG. 1). The RNA region is the hybridization binder for capturing the target RNA and modified with 2′-methylation (Novopashina et al., Nucleosides, Nucleotides and Nucleic Acids, 24, 527 (2005); Majlessi et al., Nucleic Acids Res, 26, 2224 (1998)) to increase the RNA probe stability against RNases. The DNA region serves as a template for RNase H digestion and DNA polymerase extension. Thus, the functionalized RNA probes (3′-NH₂-RNA-DNA-5′) are designed to construct the RNA microchip (or microarray) via their immobilization on the chip. The 3′-NH₂ groups of the probes enable their immobilization on the surface. For example, the probes can be immobilized on a 1, 4-phenylene diisothiocyanate (PDITC) functionalized surface via N-hydroxysuccinimide (NHS) displacement (FIG. 4) (Benters et al., ChemBioChem, 2, 686 (2001); Wittmann and Alegret, Immobilisation of DNA on chips, Springer Verlag (2005)).

To demonstrate the RNA microchip, a perfectly matched probe (M in FIG. 2A; SEQ ID NO:11) and mismatched probes (Mis) were designed for a RNA sequence. These four RNA probes were immobilized on the chip (FIG. 2). The probes were spotted on the surface by using a microarrayer generating a spot size of approximately 75μ.

After preparing the chips, several control experiments were performed. It was observed that the absence of target RNA (FIG. 2B) did not yield any signal. Also the inclusion of dNTPs (FIG. 2C) or the absence of biotin-labeled dNTPs (FIG. 2D) at Klenow extension step did not yield any signal. Only in the presence of both RNAs and biotinylated dNTPs on the microchip immobilized with RNA probes were the desired signals were observed (FIG. 2E). Because the processes of the hybridization, RNase H digestion, and DNA polymerase extension depend on the base-pairing, the overall detection specificity is attributed to these three steps: The hybridization, digestion, and extension. It was discovered that these three layers (or steps) of discrimination can help achieve high detection specificity. Surprisingly, the RNA microchip offers the high specificity at ease. Several RNA probes (see list of Dengue oligonucleotide library probes below) were designed for detecting dengue virus RNA (DV RNA). As demonstrated in FIG. 2F, only the perfectly-matched probe (M) was able to detect the DV RNA, while the single-nucleotide mis-matched and other probes (Mis-1, -2 and -3) did not. This experiment indicates the single-nucleobase differentiation with the RNA microchip. It appears that the combination of the three discriminations can easily offer single-nucleotide discrimination, without strictly tuning the hybridization conditions.

FIG. 3 shows the simultaneous detection of multiple target RNAs. The target RNAs only hybridized to the corresponding probes immobilized on the microchip. For instance, when the BA RNA was included in the sample, the Ag-NP only deposited at the BA probe sites (FIG. 3A), indicating the presence of the BA RNA. When both the BA and lacZ RNAs were included in the sample, the Ag-NP deposited at both of their probe sites (FIG. 3B), indicating the presence of the BA and lacZ RNAs. When more target RNAs were included in the sample, more corresponding probe sites (FIG. 3B-D) were deposited with Ag-NPs, forming arrays of dark spots and indicating the presence of the corresponding RNAs. These arrays of the RNA-detection signals were detectable by naked eye or with assistance of a magnifying glass. These experiments reveal that the disclosed RNA microchip technology can be used to detect multiple RNA targets simultaneously.

Detection of an individual mRNA in a total RNA was also demonstrated. When E. coli is induced with IPTG (Isopropyl β-D-1-thiogalactopyranoside) to express LacZ mRNA (>2300 nt.) and beta-galactosidase, an RNA microchip immobilized with the LacZ RNA probe was used to specifically detect the expressed LacZ mRNA in the total RNA (Chip II in both FIGS. 3E and 3F). To confirm the specificity of LacZ mRNA detection, glucose was used to suppress the expression of LacZ mRNA. Neither LacZ mRNAs nor non-specific RNAs were shown on the chip (Chip III in both FIGS. 3E and 3F), although thousands of different RNAs are present in E. coli (Rhodius et al., Annu Rev Microbiol 56:599-624 (2002)). These experimental results have demonstrated that our RNA microchip is specific and able to selectively detect individual RNA in a biological sample. In addition, a simple and rapid strategy for RNA sample preparation was demonstrated. The RNA sample can be prepared (in 5 min) by directly treating biological samples (such as E. coli) with NaOH (400 mM) for 3 min at 95° C. After neutralization and centrifugation, the RNA sample (supernatant) is ready for the chip hybridization. The NaOH treatment will fragment biological RNAs (such as mRNAs, normally several thousand nucleotides in length) into short pieces (as short as 15-25 nt.), which largely facilitate the target RNA hybridization and detection on the microchip (Spencer, L. Lin, C. F. Chiang, Z. Peng, P. Hesketh, J. Salon, Z. Huang, ChemBioChem 11:1378-1382 (2010)). With this rapid sample preparation strategy, LacZ mRNA can be selectively detected (FIG. 3F).

Detection sensitivity was also demonstrated. As shown in FIG. 5A, various quantities of target RNA were examined with the microchip. It was demonstrated that the RNA microchip can detect RNA up to the low fmole level (10⁻¹⁵). Significantly, it was demonstrated that the first three steps (RNA hybridization, RNase H digestion and DNA polymerase extension) can be consolidated into one step (FIGS. 5B and 5C), which further simplifies the detection system. The entire detection, from the beginning of the sample preparation to the end of the detection, can be accomplished in 45 min (FIGS. 3F, 5D and 5E). Furthermore, a decrease of the bacterial RNA was observed with the RNA microchip (FIG. 5E) after treating E. coli with 70% ethanol, which kills the bacteria. This is consistent with RNA rapid degradation in dead microbes due to RNA synthesis termination and enzymatic hydrolysis. Thus, the disclosed method can be useful in monitoring life or death of bacteria during disinfection treatments and infectious disease outbreaks.

Table 1 shows an example of rapid detection optimization. There are several steps that were optimized in a stepwise manner to develop a rapid RNA microchip. Silver staining was accomplished in 5-20 minutes and picture was taken in 0.6 s using CCD camera.

TABLE 1

| RNA | | Klenow | Enzymatic | Gold | |
|---|---|---|---|---|---|
| Hybridization | RNase H | incorporation | label | Nano- | Total |
| (min) | (min) | (min) | (min) | particles | (min) |
| 15 | 15 | 30 | 45 | 60 | 165 |
| 10 | 10 | 10 | 45 | 60 | 135 |
| 10 | 10 | 10 | 45 | 40 | 115 |
| 10 | 10 | 10 | 45 | 30 | 105 |
| 10 | 10 | 10 | 45 | 20 | 95 |
| 5 | 5 | 5 | 45 | 40 | 100 |
| 5 | 5 | 5 | 45 | 20 | 80 |
| 5 | 5 | 5 | 45 | 15 | 75 |
| 5 | 5 | 5 | 45 | 10 | 70 |
| 5 | 5 | 5 | 45 | 5 | 65 |
| 5 | 5 | 5 | 20 | 20 | 55 |
| 5 | 5 | 5 | 15 | 20 | 50 |
| 5 | 5 | 5 | 15 | 15 | 45 |
| 5 | 5 | 5 | 15 | 10 | 40 |
| 5 | 5 | 5 | 15 | 5 | 35 |

1. Materials and Methods

i. Oligonucleotide Design and Synthesis

The native RNAs were purchased from Integrated DNA technologies (IDT). The hybrid probes were designed and synthesized by using solid phase phosphoramidite chemistry on a 3400 DNA/RNA synthesizer (Applied Biosystems) and were purified by polyacrylamide amide gel electrophoresis. RNA probes consist of the RNA region and the 5′-DNA region (and a 3′-DNA region for some probes). The RNA region is modified with 2′ methylation is the hybridization binder for capturing the target RNA. The 3′-DNA region serves as a template for the RNase H digestion. The 5′-DNA region serves as a template for the RNase H digestion and DNA polymerase extension. The 3′-NH₂-group serves as the linker for the microchip immobilization. Thus, the functionalized RNA probes (3′-NH₂-RNA-DNA-5′ or 3′-NH₂-DNA-RNA-DNA-5′) are designed to construct the RNA microchip (or microarray) via their immobilization on the microchip.

ii. Pathogenic Oligonucleotides

LacZ RNA: E. coli LacZ mRNA (724-748 nt):
(SEQ ID NO: 1)
5′-AUGUGGAUUGGCGAUAAAAAACAA-3′
LacZ probe:
(SEQ ID NO: 2)
5′-d(GTTGTTTTTT)-2′-O-Me-RNA(AUCGCCAAUCCACAU)-
d(CTGTGAAAGA)-NH2-3′
BA RNA: Bacillus anthracis RNA (lethal factor
mRNA, 855-892 nt):
(SEQ ID NO: 3)
5′-AUCUUUAGAAGCAUUAUCUGAAGAUAAGAAAAAAA-3′
BA probe:
(SEQ ID NO: 4)
5′-d(GATTTTTTT)-2′-O-Me-RNA(CUUAUCUUCAGAUAA)-
d(TGCTTCTAAAGAT)-NH₂-3′
BF RNA: Bird flu or Avian Influenza (H5N1) RNA
[matrix protein 1 (M1) mRNA (692-729 nt)]:
(SEQ ID NO: 5)
5′-AAUCUUCUUGAAAAUUUGCAGACCUACCAAAAACGA-3′
BF probe:
(SEQ ID NO: 6)
5′-d(TCGTTTTT)-2′-O-Me-(GGUAGGUCUGCAAAAUUUU)-
d(CAAGAAGATT)-NH₂-3′
AF RNA: Avian flu (H5N1) RNA [matrix protein
(M2) mRNA (838-872 nt)]:
(SEQ ID NO: 7)
5′-CAU UUAUCGUCGC CUUUAAAUACG GUUUG AAAAGAG-3′
AF Probe:
(SEQ ID NO: 8)
5′-d(CTCTTTT)-2′-O-Me-(CAAACCGUAUUUAAG)-
d(GCGACGATAAATG)-NH₂-3′
Biotin-labeled DNA (LacZ DNA):
(SEQ ID NO: 9)
3′-NH₂-AGAAAGTGTCTACACCTAACCGCTATTTTTTGTTG-
biotin-Cy3-5′

iii. Dengue Oligonucleotide Library for Specificity Studies Obtained from Dengue RNA Genome

DENV-1 RNA (Accession no.: U88536.1;
10484-10504 nt):
(SEQ ID NO: 10)
5′-GGAAGCUGUACGC-AUGGGGUA-3′
DENV-1 Probe: (Accession no.: U88536.1;
10484-10504 nt):
(SEQ ID NO: 11)
5′-TACCCCAT-[2′-O-Me-r(GCGUACAGCUUCC)]-NH₂-3′
DENV-2 Probe: (Accession no.: U87411.1;
10470-10490 nt):
(SEQ ID NO: 12)
5′-TACGCCAT-[2′-O-Me-r(GCGUACAGCUUCC)]-NH₂-3′
DENV-3 Probe: (Accession no.: AY099336.1;
10457-10477 nt):
(SEQ ID NO: 13)
5′-TACACCGT-[2′-O-Me-r(GCGUACAGCUUCC)]-NH₂-3′
DENV-4 Probe: (Accession no.: AF326825.1;
10393-10413 nt):
(SEQ ID NO: 14)
5′-TATGCCAC-[2′-O-Me-r(GCGUACAGCUUCC)]-NH₂-3′

iv. Photographic/Imaging System

The arrays on RNA microchips were visible to naked eye (or with assistance of a magnify glass), and we used a photographing/imaging system to document the array pictures. The photographic/imaging system consisted of a microscope (Nikon Eclipse 80i) equipped with a cooled charge-coupled device camera (CCD, VersArray System; Princeton Instruments, Princeton, N.J.), and it was controlled by an Image-Pro Plus 6.1 computer software for image acquisition, processing and analysis (Media cybernetics, Inc., Silver Spring, Md.). The detection was performed using 2×2 images binning for an average of 0.6 s, 2× lens.

v. Microchip Activation

The glass chips (0.5×0.5 cm) were first degreased in CH₂CL₂ (Sigma Aldrich, St. Louis, Mo.) with gentle shaking for 30 minutes, The chips were subsequently cleaned in concentrated sulfuric acid (Sigma Aldrich, St. Louis, Mo.) for 45 minutes with shaking. The chips were rinsed with de-ionized water several times until the last wash reached to pH 7.0 and they were dried naturally. The silylation was done by incubating the chips for 45 minutes with shaking in a solution of 3% 3-aminopropyltriemethoxysilane (APTS; Sigma Aldrich, St. Louis, Mo.) in aqueous ethanol (95% ethanol and 5% water). Once the silylation was completed, the chips were washed three times: ethanol, water and ethanol. The chips were dried naturally, followed by incubation in a solution of 1, 4-phenylene diisothiocyanate (PDITC; 10 mM; Sigma Aldrich, St. Louis, Mo.) and 1% pyridine (Fluka) in CH₂Cl₂ for 2 hours. The chips were then washed three times with CH₂Cl₂, dried naturally, and stored in a desiccator at room temperature. A step-wise representation of the activation process is shown if FIG. 4.

vi. RNA Probe Immobilization

The hybrid RNA probes are dissolved (100-300 μM) in sodium phosphate buffer (100 mM, pH 8.5) followed by printing them on the activated microchip using an Omnigrid Microarrayer (Cartesian Technologies Inc., Irvine, Calif.) with a stealth pin (SMP3 pin) that deposits 0.7 nL/spot. Spotted chips are incubated in a water bath at 37° C. for 30 min.

vii. Control Experiments

Different probes designed from LacZ, B. anthracis and bird flu were immobilized on the four different chips for control experiments. The four chips were then incubated with StartingBlock blocking buffer for 20 min. On all the chips, different RNAs were applied in 5×SSC buffer (20 μL, 0.75 M NaCl, 75 mM sodium citrate, pH 7.0) and incubated at 37° C. for 15 min except for chip A. The unbound RNAs were washed twice with 1× PBS buffer (137 mM NaCl, 2.7 mM KCl, 4.3 mM Na₂HPO₄, pH 7.4)). The chips were then incubated with RNase H (0.5 μL, 5000 U mL-1) in RNase H buffer (20 μL, 1× New England Biolabs, Ipswich, Mass.) followed by incubation at 37° C. for 15 minutes. The digested RNAs were then washed twice with 1× PBS buffer followed by incubation with Klenow large fragment (0.5 μL, 5000 U mL-1) in Klenow buffer (20 μL, 1× New England Biolabs, Ipswich, Mass.) and biotin labeled dATP (1 μL, 0.4 mM, Invitrogen, Carlsbad, Calif.) on chips A and D while chip C had dNTPs applied to it. The chips were incubated at 37° C. for 30 minutes followed by washing with 1× PBS buffer. The chips were incubated with gold labeled streptavidin (4 μL) in gold conjugate dilution buffer (100 μL, KPL, Inc, Gaithersburg, Md.) for 1 hour followed by washing with 1× PBS buffer. Subsequently, 100 μL silver enhancing solution was applied until dark spots were observed. The chips were washed with a lot of water to stop further silver deposition and air dried. The pictures were taken using CCD camera.

viii. Selectivity and Simultaneous Detection of Multiple Pathogens

Different probes designed from LacZ, B. anthracis and bird flu were immobilized on the chip for selectivity and simultaneous detection studies. The chips were then incubated with StartingBlock blocking buffer for 20 min. Subsequently, the chips were flooded with different RNAs in 5×SSC buffer and incubated at 37° C. for 15 min. The unbound RNAs were washed twice with 1× PBS buffer. The chips were then incubated with RNase H (0.5 μL, 5000 U mL-1) in RNase H buffer (20 μL, 1× New England Biolabs, Ipswich, Mass.) followed by incubation at 37° C. for 15 minutes. The digested RNAs were then washed twice with 1× PBS buffer followed by incubation with Klenow large fragment (0.5 μL, 5000 U mL-1) in Klenow buffer (20 μL, 1× New England Biolabs, Ipswich, Mass.) and biotin labeled dATP (1 μL, 0.4 mM, Invitrogen, Carlsbad, Calif.). The chips were incubated at 37° C. for 30 minutes followed by washing with 1× PBS buffer. The chips were incubated with gold labeled streptavidin (4 μL) in gold conjugate dilution buffer (100 μL, KPL, Inc, Gaithersburg, Md.) for 1 hour followed by washing with 1× PBS buffer). Subsequently, 100 μL silver enhancing solution was applied until dark spots were observed. The chips were washed with a lot of water to stop further silver deposition and air dried. The pictures were taken using CCD camera.

ix. Dengue Specificity on the Chip

Different probes were designed from different section of dengue viral (DENV) RNA for specificity studies. The four dengue serotypes probes were spotted at 100 μM and incubated at 37° C. for 30 minutes. Afterward, the chips were incubated with StartingBlock (TBS, Pierce, Rockford, Ill.) blocking buffer for 20 min. The chip was then flooded with DENV-1 RNA in 5×SSC buffer. The chip was incubated at 65° C. for 15 min. The unbound RNA was washed twice with 1× PBS. The chip was then incubated with RNase H (0.5 μL, 5000 U/mL) in RNase H buffer (20 μL, 1× New England Biolabs, Mass.) followed by incubation at 65° C. for 15 minutes. The digested RNAs were then washed thrice with 1× PBS buffer followed by incubation with Klenow large fragment (0.5 μL, 5000 U/mL) in Klenow buffer (20 μL, 1× New England Biolabs, Mass.) and biotin labeled dNTPs (1 μL, 0.4 mM, Invitrogen, Carlsbad, Calif.), The chip was incubated at 65° C. for 30 minutes followed by washing three times with 1× PBS buffer. Next, the chip was incubated with gold labeled streptavidin (4 μL) in gold conjugate dilution buffer (100 μL, KPL, Inc, Gaithersburg, Md.) followed by three times washing with 1× PBS. Subsequently, 100 μL silver enhancing solution (1:1, BB International, Cardiff, CF 14 5DX, UK) was applied until dark spots were observed. The chip was washed with water to stop further silver deposition on the gold nanoparticles. Upon drying the pictures were taken using CCD camera.

x. Sensitivity of RNA Detection on the RNA Microchip

The chips were immobilized with LacZ probe for 30 minutes after which they were incubated with TBS blocking buffer for 20 min. The chips were then flooded with LacZ RNA at different concentrations in 5×SSC buffer and incubated at 37° C. for 15 min. The unbound RNA was washed twice with 1× PBS buffer. The chips were then incubated with RNase H (0.5 μL, 5000 U mL-1) in RNase H buffer (20 μL, 1× New England Biolabs, Ipswich, Mass.) followed by at 37° C. for 15 minutes. The digested RNAs were then washed twice with 1× PBS buffer followed by incubation with Klenow large fragment (0.5 μL, 5000 U mL-1) in Klenow buffer (20 μL, 1× New England Biolabs, Ipswich, Mass.) and biotin labeled dATP (1 μL, 0.4 mM, Invitrogen, Carlsbad, Calif.). The chips were incubated at 37° C. for 30 minutes followed by washing with 1× PBS buffer. The chips were then incubated with gold labeled streptavidin (4 μL) in gold conjugate dilution buffer (100 μL, KPL, Inc, Gaithersburg, Md.) followed by washing with 1× KPL wash buffer. Subsequently, 100 μL silver enhancing solution was applied until dark spots were observed. The chips were washed with a lot of water to stop further silver deposition. Upon air drying the pictures were taken using CCD camera.

xi. Rapid Detection

Three different probes were immobilized on the chip: biotin labeled LacZ probe, LacZ probe and BA probe. After immobilization, the chip was flooded with LacZ RNA sample at different concentrations in 5×SSC buffer and incubated at 37° C. for 5 min. The unbound RNAs were washed twice with 1× PBS buffer. Next, the chip was incubated with RNase H (0.5 μL, 5000 U/mL) in RNase H buffer (20 μL, 1× New England Biolabs, Ipswich, Mass.) followed by incubation at 37° C. for 5 minutes. The digested RNAs were then washed twice with 1× PBS buffer followed by incubation with Klenow large fragment (0.5 μL, 5000 U/mL) in Klenow buffer (20 μL, 1× New England Biolabs, MA) and biotin labeled dATP (1 μL, 0.4 mM, Invitrogen, Carlsbad, Calif.). The chip was incubated at 37° C. for 5 minutes followed by washing with 1× PBS buffer. The chip was incubated with gold labeled streptavidin (4 μL) in gold conjugate dilution buffer (100 μL, KPL, Inc, Gaithersburg, Md.) at 37° C. for 10 minutes followed by washing with 1× PBS buffer. Subsequently, 100 μL silver enhancing solution was applied and incubated at 37° C. for 15 min upon which the dark spots were observed. The chip was washed with water to stop further silver deposition and air dried. The pictures were taken using CCD camera. Detection was achieved in less than 1 hour.

xii. RNA Sample Preparation from E. Coli Total RNA for RNA Microchip Detection

10 mL Luria Broth (LB, Sigma Aldrich, St. Louis, Mo.) was inoculated with bacteria (E. coli K-12 MG1655. E. coli cells; 10 μL cell culture) was incubated at 37° C. overnight under shaking at 220 RPM. The overnight culture was then added to 250 mL Erlenmeyer flask containing sterilized LB (100 mL), followed by splitting (50 mL each) and culturing for 1 hour at 37° C. Isopropyl β-D-1-thiogalactopyranoside (IPTG, Sigma Aldrich, St. Louis, Mo.) was added to one flask (final concentration 1 mM). To the other, D-(+) glucose (Sigma, St. Louis, Mo.) was added (final concentration 1 mM). After both cultures were incubated at 37° C. for 4 hours, the cells were then harvested by centrifugation. The cell pellets were stored at −80° C. to be used later for RNA extraction and isolation. Total RNA of E. coli was extracted and purified according to MasterPure Complete RNA Purification Kit (Epicenter Biotechnologies Madison, Wis.), and the isolated total RNA was dissolved in TE buffer [10 mM Tris-HCl (pH 7.5), 1 mM EDTA]. The quantity of the total RNA was determined by measuring absorbance using UV-Visible spectrophotometer (Varian Inc., Santa Clara, Calif.).

RNA fragmentation was carried out by heating, alkaline- and/or metal-catalyzed fragmentation. For alkaline-catalyzed fragmentation, total RNA (0.1-10 μL, 3 μg/μL) was digested with sodium hydroxide (NaOH, final concentration 50-500 mM) at 95° C. for a few minutes. The hydrolysis reaction was neutralized and quenched by adding acetic acid, which offers the prepared RNA sample ready for the microchip hybridization and detection. For metal ion-catalyzed fragmentation, the total RNA was heated with magnesium chloride (MgCl₂, final concentration 10 mM) and zinc chloride (ZnCl₂, final concentration 10 mM) in Tris-HCl (pH 7.4, 25 mM) at 95° C. for 5 min. After cooling down, 5×SSC was added to the fragmented total RNA, which offers the prepared RNA sample ready for the microchip hybridization and detection.

xiii. Rapid RNA Sample Preparation from E. Coli by direct NaOH Treatment

E. coli pellet (such as from 1 mL LB culture) is treated with NaOH (20 μL, 400 mM) for 3 min at 95° C., followed by neutralization (adding 2 μL, 4 M acetic acid) and centrifugation (10,000 rpm) for 1 min to remove the precipitates. The supernatant is the prepared RNA sample for the microchip hybridization and detection. The RNA sample preparation can be accomplished within 5 min.

xiv. General Protocol for Direct and Rapid RNA Detection on the RNA Microchip

RNA samples (20 μL) dissolved in 5×SSC buffer (0.75 M NaCl, 75 mM sodium citrate, pH 7.0) were added onto microchips immobilized with designed RNA probes, followed by incubation and hybridization at 37° C. for 5-15 min. The unbound RNAs were removed by washing twice with 1× PBS buffer (137 mM NaCl, 2.7 mM KCl, 11.8 mM Na—PO₄, pH 7.4). The chips were then incubated with RNase H (2.5 U, New England Biolabs, Ipswich, Mass.) in 1× RNase H buffer (20 μL) at 37° C. for 5-15 minutes. The digested RNAs were removed by washing the chips twice with 1× PBS buffer, followed by incubation with Klenow (2.5 U, New England Biolabs, Ipswich, Mass.) and biotin-labeled dNTPs (each 0.4 mM final concentration; Invitrogen, Carlsbad, Calif.) in 1× Klenow buffer (20 μL) at 37° C. for 5-20 min. After washing the chips with 1× PBS buffer twice, the chips were incubated with streptavidin-labeled gold nanoparticle (4 μL, KPL, Inc, Gaithersburg, Md.) in its buffer (100 μL) for 5-30 min, followed by washing the chips with 1× PBS buffer twice. Subsequently, the silver enhancing solution (100 μL of 1:1 mixture; BB International, Cardiff, CF14 5DX, UK) was applied on the chips, and the chips were incubated at 37° C. for 5-30 min until the dark spot arrays were formed on the surface of the chips. The chips were washed with water to quench the silver deposition and dried naturally. The spot arrays on chips were observed by naked eye (or with assistance of a magnifying glass) for the direct and rapid RNA detection (entire detection time 45 min, including RNA sample preparation). The spot-array images on the surfaces of the RNA microchips were also photographed and analyzed with a microscope-CCD-camera imaging system.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a chimeric probe” includes a plurality of such chimeric probes, reference to “the chimeric probe” is a reference to one or more chimeric probes and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different specific binding molecules does not indicate that the listed specific binding molecules are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of detecting RNA in a sample, the method comprising:
(a) bringing into contact the sample and a probe array, wherein the probe array comprises one or more chimeric probes, wherein the chimeric probes comprise a DNA region and a RNA region, wherein the DNA region and the RNA region are contiguous, wherein the DNA region is 5′ of the RNA region, wherein if the sample contains an RNA molecule complementary to a nucleotide sequence of at least one of the chimeric probes, the RNA molecule will hybridize with the complementary chimeric probe;
(b) bringing into contact the probe array and a ribonuclease specific for RNA/DNA hybrids, wherein the portion of the RNA molecule hybridized to the DNA region of the chimeric probe is degraded;
(c) bringing into contact the probe array, labeled nucleotides, and a nucleic acid polymerase capable of extending a RNA strand using a DNA template and capable of incorporating the labeled nucleotides in the extension from the RNA strand, wherein the hybridized RNA molecule is extended to form an extended nucleic acid strand, wherein at least one labeled nucleotide is incorporated into the extended nucleic acid strand, wherein each of the labeled nucleotides comprises a first label; and
(d) detecting the labeled nucleotides in the extended nucleic acid strand by bringing into contact the probe array and a label conjugate, wherein the label conjugate comprises a specific binding molecule and a second label, wherein the specific binding molecule binds to the first label, wherein the labeled nucleotides in the extended nucleic acid strand are detected by detecting the second label, wherein the second label is detected by bringing into contact the probe array and a detection conjugate, wherein the detection conjugate comprises an aggregator, and wherein the aggregator mediates aggregation of detection conjugates on the label conjugate,
wherein detection of the labeled nucleotides in the extended nucleic acid strand indicates the presence of the RNA molecule in the sample.

2. The method of claim 1, wherein detection conjugate associates with or binds to the second label.

3. The method of claim 1, wherein the second label mediates or allows aggregation of the detection conjugates on and at the label conjugate.

4. The method of claim 1, wherein the detection conjugate includes or generates a signal.

5. The method of claim 1, wherein the detection conjugate includes multiple copies of a label.

6. The method of claim 1, wherein multiple detection conjugates associate with or bind to the second label.

7. The method of claim 1, wherein the detection conjugate self-aggregates.

8. The method of claim 1, wherein the first label comprises biotin, wherein the specific binding molecule comprises streptavidin, wherein the second label comprises gold, and wherein the aggregator comprises silver.

9. The method of claim 1, wherein steps (a), (b), and (c) are carried out simultaneously.

10. The method of claim 1, wherein the label conjugate comprises a gold nanoparticle conjugated to streptavidin.

11. The method of claim 1, wherein the detection conjugate comprises a silver nanoparticle.

12. The method of claim 1, wherein the ribonuclease is RNase H, and wherein the nucleic acid polymerase is Klenow fragment of DNA polymerase.

13. The method of claim 1, wherein the probe array further comprises a solid state substrate, and wherein the chimeric probes are immobilized on the solid state substrate.

14. The method of claim 13, wherein the probe array comprises more than one chimeric probe, wherein each chimeric probe is immobilized in a different location of the solid state substrate, wherein each chimeric probe has a different nucleotide sequence, wherein each chimeric probe is complementary to a different RNA molecule, wherein the location on the solid state substrate where the labeled nucleotides in the extended nucleic acid strand are detected indicates the identity of the RNA molecule detected.

15. The method of claim 1, wherein the chimeric probes are stabilized nucleic acids.

16. The method of claim 15, wherein the RNA region is comprised of 2′-O-methyl nucleotides.

17. The method of claim 1, wherein one or more of the chimeric probes are complementary to a nucleotide sequence characteristic of a virus, a bacteria, or a microbe.

18. The method of claim 1, wherein one or more of the chimeric probes further comprise a second DNA region, wherein the second DNA region is 3′ of the RNA region.

19. The method of claim 18, wherein the DNA region and the RNA region are contiguous.

20. The method of claim 1, wherein the chimeric probes further comprise a 3′-linking group, wherein the linking group mediates immobilization of the chimeric probes.

21. The method of claim 20, wherein the 3′-linking group is an amino group.

22. A kit comprising a probe array, labeled nucleotides a label conjugate, and a detection conjugate,
wherein the probe array comprises one or more chimeric probes, wherein the chimeric probes comprise a DNA region and a RNA region, wherein the DNA region and the RNA region are contiguous, wherein the DNA region is 5′ of the RNA region, and wherein a nucleotide sequence of the chimeric probes are complementary to a nucleotide sequence of a RNA molecule of interest,
wherein each of the labeled nucleotides comprises a first label, wherein the label conjugate comprises a specific binding molecule and a second label, and wherein the specific binding molecule binds to the first label,
wherein the detection conjugate comprises an aggregator, and wherein the aggregator mediates aggregation of detection conjugates on the label conjugate.

23. The kit of claim 22, wherein detection conjugate can associate with or bind to the second label.

24. The kit of claim 22, wherein the second label can mediate or allow aggregation of the detection conjugates on and at the label conjugate.

25. The kit of claim 22, wherein the detection conjugate includes or can generate a signal.

26. The kit of claim 22, wherein the detection conjugate includes multiple copies of a label.

27. The kit of claim 22, wherein multiple detection conjugates can associate with or bind to the second label.

28. The kit of claim 22, wherein the detection conjugate can self-aggregate.

29. The kit of claim 22, wherein the probe array further comprises a solid state substrate, and wherein the chimeric probes are immobilized on the solid state substrate.

30. The kit of claim 22, wherein the probe array comprises more than one chimeric probe, wherein each chimeric probe is immobilized in a different location of the solid state substrate, wherein each chimeric probe has a different nucleotide sequence, and wherein each chimeric probe is complementary to a different RNA molecule.

31. The kit of claim 22, wherein the chimeric probes are stabilized nucleic acids.

32. The kit of claim 31, wherein the RNA region is comprised of 2′-O-methyl nucleotides.

33. The kit of claim 22, wherein one or more of the chimeric probes are complementary to a nucleotide sequence characteristic of a virus, a bacteria, or a microbe.

34. The kit of claim 22, wherein one or more of the chimeric probes further comprise a second DNA region, wherein the second DNA region is 3′ of the RNA region.

35. The kit of claim 34, wherein the DNA region and the RNA region are contiguous.

36. The kit of claim 22, wherein the chimeric probes further comprise a 3′-linking group, wherein the linking group mediates immobilization of the chimeric probes.

37. The method of claim 36, wherein the 3′-linking group is an amino group.

38. A probe array comprising one or more chimeric probes, wherein the chimeric probes comprise a DNA region and a RNA region, wherein the DNA region and the RNA region are contiguous, wherein the DNA region is 5′ of the RNA region, and wherein a nucleotide sequence of the chimeric probes are complementary to a nucleotide sequence of a RNA molecule of interest.

39. The probe array of claim 38, wherein the probe array further comprises a solid state substrate, and wherein the chimeric probes are immobilized on the solid state substrate.

40. The probe array of claim 38, wherein the probe array comprises more than one chimeric probe, wherein each chimeric probe is immobilized in a different location of the solid state substrate, wherein each chimeric probe has a different nucleotide sequence, and wherein each chimeric probe is complementary to a different RNA molecule.

41. The probe array of claim 38, wherein the chimeric probes are stabilized nucleic acids.

42. The probe array of claim 41, wherein the RNA region is comprised of 2′-O-methyl nucleotides.

43. The probe array of claim 38, wherein one or more of the chimeric probes are complementary to a nucleotide sequence characteristic of a virus, a bacteria, or a microbe.

44. The probe array of claim 38, wherein one or more of the chimeric probes further comprise a second DNA region, wherein the second DNA region is 3′ of the RNA region.

45. The probe array of claim 44, wherein the DNA region and the RNA region are contiguous.

46. The probe array of claim 38, wherein the chimeric probes further comprise a 3′-linking group, wherein the linking group mediates immobilization of the chimeric probes.

47. The probe array of claim 46, wherein the 3′-linking group is an amino group.
